(19) United States
(12) Patent Application Publication
(10) Pub. No.: US 2025/0123267 A1
(43) Pub. Date: Apr. 17, 2025

(54) INHIBITORS OF MICROTUBULE POLYGLUTAMYLATION IN MITOSIS FOR USE AS A MEDICAMENT

(71) Applicants: FUNDACIÓ CENTRE DE REGULACIÓ GENÒMICA, Barcelona (ES); INSTITUCIÓ CATALANA DE RECERCA I ESTUDIS AVANÇATS, Barcelona (ES)

(72) Inventors: Isabelle Vernos, Barcelona (ES); Ivan Zadra, Barcelona (ES)

(73) Assignees: FUNDACIÓ CENTRE DE REGULACIÓ GENÒMICA, Barcelona (ES); INSTITUCIÓ CATALANA DE RECERCA I ESTUDIS AVANÇATS, Barcelona (ES)

(21) Appl. No.: 18/711,500

(22) PCT Filed: Nov. 17, 2022
(86) PCT No.: PCT/EP2022/082269

§ 371 Date: May 17, 2024

(30) Foreign Application Priority Data
Nov. 18, 2021 (EP) 21383045.8

(51) Int. Cl. G01N 33/50; G01N 33/573; G01N 33/574; G01N 33/68

(52) U.S. Cl. CPC G01N 33/5011; G01N 33/573; G01N 33/574; G01N 33/6848; G01N 2333/9015; G01N 2440/34

(57) ABSTRACT

The present invention refers to inhibitors of microtubule polyglutamylation in mitosis for use as a medicament, preferably in the treatment of diseases benefiting from the inhibition of microtubule polyglutamylation in mitosis, most preferably in the treatment of cancer.

### FIELD OF THE INVENTION

The present invention refers to inhibitors of microtubule polyglutamylation in mitosis, preferably TTLL11 inhibitors, for use as a medicament, preferably in the treatment of diseases benefiting from the inhibition of microtubule polyglutamylation in mitosis, most preferably in the treatment of cancer.

### DESCRIPTION OF THE INVENTION

### Brief Description of the Invention

During mitosis, spindle microtubules (MTs) are polyglutamylated but the enzyme driving this post-translational modification and its functional implications in mitosis have not been elucidated. Here we identify TTLL11 as an enzyme that catalyzes the polyglutamylation of the spindle MTs in mitosis. Using a combination of approaches in Hela cells and zebrafish embryos we show that polyglutamylation defines spindle MT dynamics preventing chromosome segregation errors beyond the spindle assembly checkpoint. We also show that TTLL11 is consistently downregulated in human tumors, opening the exciting possibility that reduced spindle MT polyglutamylation could play an important role in aneuploidy, one of the most salient features of cancer cells.

So, the first embodiment of the present invention refers to inhibitors of microtubule polyglutamylation in mitosis for use as a medicament.

In a preferred embodiment of the present invention, the inhibitors of microtubule polyglutamylation in mitosis are TTLL11 inhibitors.

In a preferred embodiment of the present invention, the inhibitors of microtubule polyglutamylation in mitosis are used in the treatment of diseases benefiting from the inhibition of microtubule polyglutamylation in mitosis.

In a preferred embodiment of the present invention, the inhibitors of microtubule polyglutamylation in mitosis are used in the treatment of cancer.

Alternatively, the present invention refers to a method for treating a patient, preferably a patient suffering from a disease benefiting from the inhibition of microtubule polyglutamylation in mitosis, which comprises administering a therapeutically effective dose or amount of inhibitors of microtubule polyglutamylation in mitosis, or a pharmaceutical composition comprising thereof.

In a preferred embodiment of the present invention, the patient to be treated is a patient suffering from cancer.

In a preferred embodiment of the present invention, the inhibitors of microtubule polyglutamylation in mitosis are TTLL11 inhibitors.

In a preferred embodiment of the present invention, the inhibitor is a biologic agent or a chemical compound.

In a preferred embodiment of the present invention, the inhibitor is a small molecule or a siRNA.

The second embodiment of the present invention refers to a pharmaceutical composition comprising an inhibitor of microtubule polyglutamylation in mitosis and, optionally, pharmaceutically acceptable carriers or excipients.

In a preferred embodiment of the present invention, the pharmaceutical composition comprises a TTLL11 inhibitor and, optionally, pharmaceutically acceptable carriers or excipients.

In a preferred embodiment of the present invention, the pharmaceutical composition comprises an inhibitor selected from a biologic agent or a chemical compound and, optionally, pharmaceutically acceptable carriers or excipients.

In a preferred embodiment of the present invention, the pharmaceutical composition comprises an inhibitor selected from a small molecule inhibitor or a siRNA and, optionally, pharmaceutically acceptable carriers or excipients.

In a preferred embodiment of the present invention, the inhibitor is an ATP-competitive inhibitor.

The third embodiment of the present invention refers to an in vitro method for the diagnosis and/or prognosis of a disease benefiting from the inhibition of microtubule polyglutamylation in mitosis, which comprises assessing the level of expression of TTLL11 in a biological sample obtained from the patient, wherein the identification of a reduced level of expression of TTLL11, as compared with a pre-established level of expression measured in healthy control patients, is as indication that the patient is suffering from a disease benefiting from the inhibition of microtubule polyglutamylation in mitosis.

In a preferred embodiment the present invention refers to an in vitro method for the diagnosis and/or prognosis of cancer which comprises assessing the level of expression of TTLL11 in a biological sample obtained from the patient, wherein the identification of a reduced level of expression of TTLL11, as compared with a pre-established level of expression measured in healthy control patients, is as indication that the patient is suffering from cancer.

The fourth embodiment of the present invention refers to the in vitro use of TTLL11 for the diagnosis and/or prognosis of diseases benefiting from the inhibition of microtubule polyglutamylation in mitosis.

In a preferred embodiment, the present invention refers to the in vitro use of TTLL11 for the diagnosis and/or prognosis of cancer.

The fifth embodiment of the present invention refers to an in vitro method for screening, identifying and/or producing compounds for use as medicaments which comprises: a) Determining whether the inhibition of microtubule polyglutamylation in mitosis by the candidate compound has taken place, and b) wherein if said inhibition has taken place, it is indicative that the candidate compound may be effective as medicament.

In a preferred embodiment, the present invention refers to an in vitro method for screening, identifying and/or producing compounds for use as medicaments which comprises: a) Assessing TTLL11 enzyme activity once the candidate compound has been incubated with the TTLL11 enzyme, and b) wherein if an inhibition of TTLL11 activity is observed, it is indicative that the candidate compound may be effective as medicament.

In a preferred embodiment, the method is characterized in that it is a non-radioactive assay wherein TTLL11 activity is assessed by analysing whether additional glutamates haven been incorporated by the enzyme TTLL11 to the enzyme substrate by using an immunoassay or by mass spectrometry, wherein if no additional glutamates have been incorporated to the enzyme substrate this is an indication that the candidate compound has inhibited TTLL11 activity and may be effective as medicament.

In a preferred embodiment, the method is characterized in that the enzyme substrate is a stabilized microtubule which has been obtained by tubulin polymerization and the activity of TTLL11 enzyme is assessed by analysing whether additional glutamates haven been incorporated by the enzyme TTLL11 to the enzyme substrate by using an immunoassay.

In a preferred embodiment, the method is characterized in that the enzyme substrate is a peptide corresponding to the C-terminal region of tubulin (alpha or beta) and the activity of TTLL11 enzyme is assessed by analysing whether additional glutamates haven been incorporated by the enzyme TTLL11 to the enzyme substrate by mass spectrometry.

In a preferred embodiment, the present invention refers to a method for screening, identifying and/or producing compounds for the treatment of diseases benefiting from the inhibition of microtubule polyglutamylation in mitosis.

In a preferred embodiment, the present invention refers to a method for screening, identifying and/or producing compounds for the treatment of cancer.

For the purpose of the present invention, the following terms are defined:
The term “comprising” means including, but not limited to, whatever follows the word “comprising”. Thus, the use of the term “comprising” indicates that the listed elements are required or mandatory but that other elements are optional and may or may not be present.
The term “consisting of” means including, and limited to, whatever follows the phrase “consisting of”. Thus, the phrase “consisting of” indicates that the listed elements are required or mandatory and that no other elements may be present.
“Pharmaceutically acceptable excipient or carrier” refers to an excipient that may optionally be included in the compositions of the invention and that causes no significant adverse toxicological effects to the patient.
By “therapeutically effective dose or amount” of the pharmaceutical composition of the invention is intended an amount that, when administered to the subject, brings about a positive therapeutic response in a subject suffering from diseases benefiting from the inhibition of microtubule polyglutamylation in mitosis, preferably cancer. The exact amount required will vary from subject to subject, depending on the species, age, and general condition of the subject, the severity of the condition being treated, mode of administration, and the like. An appropriate “effective” amount in any individual case may be determined by one of ordinary skill in the art using routine experimentation, based upon the information provided herein.

### DESCRIPTION OF THE FIGURES

FIG. 1. TTLL11 localizes to the spindle and drives MT polyglutamylation in mitosis. (A) Immunofluorescence images of a HeLa metaphase spindle expressing GFP-TTLL11. Tubulin (red), anti-GFP (green) and DAPI (blue). (B) Immunofluorescence images of metaphase spindles in control and siTTLL11 cells. PolyE (green), tubulin (red) and DNA (blue). (C) Quantification of the polyE signal normalized to the total tubulin signal in control and siTTLL11 spindles. n (control)=105 cells and n (siTTLL11)=50 cells. Graph representative of N=5 independent experiments. (D) Quantification of spindle length, width and area for at least 47 cells. Graph representative of N=3 independent experiments. Scale bars, 10 μm. Error bars represent SD.

FIG. 2. siTTLL11 spindles have less dynamic MTs and mis-segregate chromosomes. (A) Confocal images of tubulin photoactivated (dark grey) close to the metaphase spindle equator (0 min) over time (min) in control and siTTLL11 cells. (B) Velocity of the poleward flux in control (n=18) and siTTLL11 (n=14) cells. (N=3) (C) Quantification of cold induced K-fiber depolymerization over time in control and siTTLL11 cells. Cells were classified into four categories as shown. Scale bar, 5 μm. (n≥40; N=3) (D) Immunofluorescence image of a metaphase cell. Chromosomes (blue), CREST (red, kinetochore) and Hec1 (green). In the magnification, the white arrow shows the measured interkinetochore distance. Scale bar, 5 μm. Zoom-in scale bar, 2 μm (E) Quantification of interkinetochore distance. n (control) ≥72 kinetochore pairs, 16 cells; n (siTTLL11) ≥82 kinetochore pairs, 18 cells. (F) Immunofluorescence images of two siTTLL11 anaphase cells with lagging chromosomes (white arrows). (G) Quantification of lagging chromosome frequency in control and siTTLL11 cells (n≥121). Graphs in B, C and D are representative from three independent experiments. Scale bar, 15 μm. All error bars are SD. p-values are based on unpaired t-test with 95% confidence.

FIG. 3. Spindle MTs polyglutamylation by TTLL11 is required for chromosome segregation fidelity in zebrafish embryos. (A) Immunofluorescence image of a zebrafish embryo (4 hpf, hours post fertilization) showing PolyE (green), tubulin (red) and DNA (blue). Scale bar, 20 μm. (B) Semi-quantitative PCR showing zfTTLL11 expression in zebrafish embryos. Lanes 1, 4 cells; 2, 8 cells; 3, 64 cells; 4, 256 cells; 5, Sphere; 6, Shield; 7, 70% epiboly; 8, 90% epiboly; 9, 24 hpf. eef1A was amplified as control. (C) Fluorescent image of a spindle in a zebrafish embryo expressing GFP-zfTTLL11. GFP, red; H2B-mCherry, blue. Scale bar, 20 μm. (D) Zebrafish embryos (36 hpf) injected at zygote stage with scrambled MO or zfTTLL11-MO (MO-1) and zfTTLL11-MO co-injected with zfTTLL11 mRNA (WT) or catalytically inactive zfTTLL11-E466G mRNA. Quantification of the phenotypes at 36 hpf. Graph representative from N=4. (n≥21). (E) Immunofluorescence image of a zebrafish embryo at sphere stage (4 hpf) showing an anaphase cell with a lagging chromosome (arrow). Frequency of anaphase cells with lagging chromosomes (N=2). Scale bar, 10 μm. (F) Still image from a time lapse recording of an anaphase cell in a zfTTLL11-MO embryo showing a lagging chromosome (white arrow). Frequency of embryos showing at least one chromosome mis-segregation event during the imaging period. (N=4; n≥4). Scale bar, 15 μm.

FIG. 4. TTLL11 expression is downregulated in tumors and correlates negatively with aneuploidy scores. (A) Normalized expression of TTLL11 in solid tissue normal and primary tumor samples across 13 different cancer types, separately and combined (PANCAN). p-values: (****)≤0.0001, (***)≤0.001, (**)≤0.01, (*)≤0.05, based on unmatched Wilcoxon Rank Sum tests. (B) Spearman correlation coefficients of normalized gene expression and sample aneuploidy scores for every gene across 13 different cancer types. PANCAN represents the median correlation coefficient for every gene across cancers. TTLL11 is highlighted in red. p-values: (*)≤0.05, (.)≤0.01, based on one-sided one-sample Z tests.

FIG. 5. Spindle MT polyglutamylation levels are reduced in cancer cells.

a—Immunofluorescence images of metaphase spindles in control and siTTLL11 hTERT-RPE1 untransformed cells, showing the PolyE signal (green), tubulin (red) and DNA (blue). Scale bars, 10 μm.

b—Immunofluorescence images of metaphase spindles in a panel of cancer cell lines as indicated. The PolyE signal (green), tubulin (red) and DNA (blue) are shown. Scale bars, 10 μm.

c—Quantification of the polyE signal normalized to the total tubulin signal in spindles from hTERT-RPE1 and cancer cells shown in b. n (RPE1)=15 cells, n (RPE1 siTTLL11)=20 cells, n (HT-29)=15 cells, n (HCT-116)=41 cells, n (MDA-MD-231)=10 cells, n (MDA-MD-468)=11 cells and n (U2O2)=17 cells. Error bars represent the SD. Source data are provided as a Source Data file.

FIG. 6. Coexpression-based analysis pinpoints the overexpression of oncogenes CCNE1 or CDC25A as a cause for the consistent downregulation of TTLL11 across tumours.

a—Differences in normalized enrichment scores (NES) (Delta NES) between the tumour and healthy coexpression signatures (x-axis) of each TTLL glutamylase enzyme correlated against the rest of the genes. NES were obtained through gene-set enrichment analysis (GSEA) and show whether a coexpression profile is significantly enriched (FDR<0.05) in genes targeted by a transcription factor (y-axis) from the ChIP-seq-based ChEA dataset 24).

b—Overlap between enriched transcription factor targets coexpressing with TTLL11 in tumours.

c—Coexpression (y-axis) between TTLL11 and enriched 119 transcription factor targets of KDM5B, ASH2L, and NANOG in tumours (x-axis). d—Normalized gene expression of TTLL11 with respect to the control (“Empty”) condition (y-axis) upon overexpression with doxycycline (“Treatment” colour) of different oncogenes (x-axis) across 4 different cell models (“Cell Line” dot shape). Each condition and treatment contains N=16 biologically independent experiments (4 for each cancer cell line). In each box plot, the median value is indicated as a horizontal line and the lower and upper bounds of the box correspond to the first and third quartiles, respectively. The upper and lower whiskers range from the corresponding box hinges to the largest value no further than 1.5 times the inter-quartile range from the hinge. All outlying data points beyond the whiskers are plotted individually.

e—Normalized expression of TTLL11 (y-axis) in healthy solid tissues and the corresponding primary tumour samples stratified by the expression of CCNE1 and CDC25A (“Sample Type” colour) across 13 different cancer types (x-axis), separately and combined (PANCAN). Statistical tests compare healthy solid tissues against either samples with high expression of CCNE1 or CDC25A (“CCNE1_high or CDC25_high”) or samples with low expression of both upstream regulators of TTLL11 (“CCNE1_low & CDC25A_low”). Number of samples per cancer type and sample type. In each box plot, the median value is indicated as a horizontal line and the lower and upper bounds of the box correspond to the first and third quartiles, respectively. The upper and lower whiskers range from the corresponding box hinges to the largest value no further than 1.5 times the inter-quartile range from the hinge. All outlying data points beyond the whiskers are plotted individually. In all the figure, nominal p-values: (****)≤0.0001, (***)≤0.001, (**)≤0.01, (*)≤0.05, based on unmatched two-sided Wilcox Rank Sum tests.

FIG. 7. Bi-allelic knockout of zfTTLL11

a—WT zebrafish embryos (36 hpf) injected at the one cell stage with the three control gRNA and Cas9 as control (−) or the three gRNA against TTLL11 and Cas9 (Cas9 3×gRNA, +). Scale bar, 1 mm.

b—Cumulative bar plot of developmental defects in the two experimental conditions as described in a: severe (III); mild (II) or no defects (I) in 36-hpf embryos from N=2 independent experiments, (≤20 embryos scored per condition). p-values are based on χ² test with a 95% confidence interval.

c—Immunofluorescence images of zebrafish Cas9 3×gRNA injected embryos (4 hpf) showing chromosome segregation defects in anaphase cells. Scale bar, 10 μm.

d—Quantification of anaphase cells with lagging chromosomes in control (−) (n=24) and Cas9 3×gRNA (+) (n=32) injected embryos, N=1. p-values values are based on a χ² test with a 95% confidence.

e—Quantification of fixed embryos showing at least one or more micronucleated cells in control (−) (n=7) and Cas9 3×gRNA (+) (n=11) embryos.

FIG. 8. Western blot of purified GST-telokins having glutamate chains of different lengths and composition as indicated. The PolyE antibody only recognizes chains of 3 or more glutamates. The anti-GST antibody was used as a loading control.

FIG. 9. Schematic representation of assay 1 of the experimental assays to screen for compounds with inhibitory activity against TTLL11. The reactivity of the PolyE antibody at each step of the assay is represented at the bottom.

Figure S1. Phylogenetic tree of TTLLs enzymes. The tree includes all the TTLL enzymes from human, mouse, zebra fish, Drosophila and C. elegans. It was developed with posterior probability algorithm setting TTLL12 as the outgroup. The TTLL11 branch is highlighted with a red shadow. The position of the glycylases is indicated with a light blue cone. Figure S2. GFP-TTLL13 localizes to the spindle. Immunofluorescence images of a HeLa metaphase spindle expressing GFP-TTLL13. Anti-GFP (green), Tubulin (red) and DAPI (blue). Scale bar, 10 μm.

Figure S3. Expression of TTLL11 and TTLL13 in human tissues and HeLa cells. (A) Expression levels of TTLL11 and TTLL13 across human tissues from GTEx illustrated as a red scale in log 2(TPM+1). (B) Relative expression of actin, TTLL11 and TTLL13 in HeLa cells detected by RT-qPCR. The plot represent means from N=3 independent experiments. Error bars represent SD.

Figure S4. TTLL11 silencing efficiency in HeLa cells. (A) Western blot analysis of control and siTTLL11 cells expressing GFP-hTTLL11. The blot was probed with an anti-GFP to visualize exogenous GFP-TTLL11, an anti-tubulin as loading control and the anti-PolyE antibody-Note that cells overexpressing GFP-TTLL11 have a higher level of polyglutamylated tubulin. Cell lysates were obtained 48h after transfection. (B) TTLL11 expression levels detected by RT-qPCR in control and TTLL11 silenced cells 48 h after transfection. Expression levels represent means from N=3 independent experiments. Error bars represent SD.

Figure S5. Schematic representation of tubulin (poly) glutamylation and the specificities of the antibodies GT335 and PolyE. Polyglutamylation is generated by a family of TTLL enzymes with different enzymatic specificities (7). Enzymes can be classified into initiating enzymes that can generate the first link between the C-terminal tubulin tails (grey amino acids) and the first glutamates of the branched glutamate chains (orange glutamate residues). Elongating enzymes, like TTLL11, add linear glutamate chains (red glutamates) onto the nascent (orange) glutamate branch chains. The monoclonal antibody GT335 specifically detects the branched structure generated by the initiating glutamylases (orange) (29). By contrast, polyE detects linear glutamate chains of more than three glutamate residues with a C-terminal carboxy group exposed (30). This antibody thus mostly detects long glutamate chains (red glutamates) generated by elongating polyglutamylases like TTLL11. Note that several glutamate residues within the C-terminal tails (grey) of both, α- and β-tubulin are modified by polyglutamylation. These differences can so far not be distinguished by GT335 or polyE.

Figure S6. MT glutamylation detected by the anti GT335 antibody in interphase and mitotic HeLa cells is not altered upon TTLL11 silencing. (A) Immunofluorescence images of interphase and mitotic control and siTTLL11 HeLa cells. Tubulin (red), GT335 (green) and DAPI (blue). Scale bars, Interphase, 20 μm; Mitosis, 10 μm. (B) Mean of normalized GT335 signal in the different conditions. Mitosis n (control)=35 and n (siTTLL11)=38. Interphase n (control)=27 and n (siTTLL11)=33. N=2. Error bars are SD. p-value are based on two-way ANOVA test with a 95% confidence interval.

Figure S7. TTLL11 silencing does not alter microtubule polyglutamylation levels in interphase. MT glutamylation levels were monitored by immunofluorescence with the anti-PolyE antibody in control and siTTLL11 interphase HeLa cells. (A) Immunofluorescence images of control and siTTLL11 interphase cells. Tubulin (red) polyE (green) and DAPI (blue). Scale bar, 20 μm. (B) Mean of normalized anti-polyE antibody signal in interphase control and siTTLL11 cells. Mitosis n (control)=56 and n (siTTLL11)=54. Interphase n (control)=14 and n (siTTLL11)=14. N=2. Scale bars, 10 m. p-values are based on two-way ANOVA test with a 95% confidence interval.

Figure S8. The level of MT polyglutamylation is reduced in TTLL11 silenced cells specifically in mitosis. Western blots of Hela cell lysates probed to detect TPX2, Tubulin and Polyglutamylated tubulin (polyE-tubulin). TPX2 peaks in G2/M and was used as a marker for G2/M synchronized cells. For quantification increasing amounts of the cell lysates were loaded for each condition as indicated on top: 1-15 μg, 2-25 μg, 3-35 μg, 4-45 μg.

Figure S9. Mitotic progression in control and siTTLL11 HeLa cells. (A) Selected frames from time lapse movies of control and siTTLL11 HeLa cells constitutively expressing H2B-mRFP/α-tubulin-GFP. Tubulin, red; DNA, green. Scale bar, 20 μm. (B) Time from NEBD to anaphase for control and siTTLL11 cells. Error bars are SD. N=3. n (control)=66 and n (siTTLL11)=46. p-values are based on unpaired t-test with 95% confidence. (C) Percentage of mitotic cells in control and TTLL11 silenced HeLa. n=at least 1000 cells for each condition. N=3.

Figure S10. Error correction is active in TTLL11 silenced cells. (A) Time lapse images of control and siTTLL11 HeLa H2B-mRFP/α-tubulin-GFP cells released from a STLC treatment. Time is in min. Tubulin, red and DNA, green. Scale bar, 10 μm. (B) Bar plot showing the time control and siTTLL11 takes to enter anaphase from STLC release. n (control)=46 and n (siTTLL11)=87. N=3. Error bars are SD. p-values are based on unpaired t-test with 95% confidence.

Figure S11. Embryonic lethality and micronuclei formation in TTLL11 morpholino injected zebrafish embryos. (A) Percentage of dead embryos over the first 4 hours of development for control and MO-1 injected embryos as well as embryos co-injected with Mo-1 and GFP-zfTTLL11 mRNA (WT). The numbers of dead/total embryos are shown above each bar. The bar plot is representative of N=3. Minimum n=29. (B) Stills from Time lapse images of control and MO-1 injected 4 hpf zebrafish embryos stably expressing H2A-mCherry (black). Orange arrowheads point to multiple micronuclei, red arrowheads point to a lagging chromosome in anaphase. Scale bar, 20 μm. (C) Stills from the time lapse images of the anaphase cell in a MO-1 injected embryo highlighted in A (black square) showing that the lagging chromosome forms a micronucleus. Scale bar, 10 μm. Time is in hh:mm:ss.

Figure S12. TTLL11 is rarely mutagenized in tumors. Mutation frequencies per kilobase for every detected gene across 13 different cancer types. PANCAN represents the median mutation frequency per kilobase for every gene across cancers. TTLL11 is highlighted in red. Mutation frequencies are reported by their predictive type of effect. (A) missense mutation, (B) nonsense mutation, (C) 3′UTR, (D) splice site, (E) frameshift deletion. p-values: (*) 0.05 (.) 0.01, based on one-sided one-sample Z tests.

Figure S13. Promoter methylation of TTLL11 does not explain the consistent transcriptomic downregulation of TTLL11 in tumours. Median beta scores for all methylation sites within the 1,500 bp upstream of TTLL11 transcription start site (y-axis) across 13 different cancer types (x-axis), separately and combined (PANCAN). p-values: (****)≤ 0.0001, (***)≤0.001, (**)≤0.01, (*)≤0.05, based on unmatched Wilcox Rank Sum tests.

Figure S14. Expression levels of TTLL11 and other TTLLs in cancer

a. Graph showing the frequency of the differential expression and the direction (e.g., upregulated or downregulated) for each TTLL in primary tumours versus unmatched healthy solid tissue samples across 13 different types of cancer (Wilcoxon rank sum test; FDR <0.05). The numbers indicate the number of types of cancer in which each TTLL is differentially expressed.

b—TTLL13 is less expressed than TTLL11 in cancer. Normalized expression of TTLL11 and TTLL13 in primary tumor samples across 13 different cancer types, separately and combined (PANCAN). p-values: ****p≤0.0001; ***p≤0.001; **p≤0.01; *p≤0.05; based on unmatched Wilcoxon rank sum tests.

Figure S15. Coexpression-based enrichment analysis of TTLL glutamylases and putative upstream regulators of TTLL11.

a. Co-expression signature between each TTLL glutamylase and the rest of the genes in samples from primary tumours (x-axis) and healthy solid tissue (y-axis). In each subpanel, Spearman correlations between the two signatures and corresponding p-values are shown.

b. Differences in normalized enrichment scores (NES) (Delta NES) between the tumour and healthy solid tissue coexpression signatures (x-axis) of each TTLL glutamylase enzyme correlated against the rest of the genes. NES were obtained through gene-set enrichment analysis (GSEA) and show whether a coexpression signature is significantly enriched (FDR <0.05) in genes from a biological process listed in the Gene Ontology database.

c. Normalized expression of genes CCNE1 (x-axis) and CDC25A (y-axis) in primary tumor samples across 13 different human cancer types (as indicated at the top of each graph). The dot colours indicate high expression levels of either CCNE1 or CDC25A (“CCNE1_high & CDC25A_high”, red), low levels of both (“CCNE_low & CDC25A_low”, yellow), or was not relevant for this classification (“N.R.”, black). In each subpanel, Spearman correlations between the two signatures and corresponding p-values are shown.

### DETAILED DESCRIPTION OF THE INVENTION

The examples set below are provided with the purpose of illustrating the invention and without the intention of limiting its scope of protection.

### Example 1. Material and Methods

### Example 1.1. Cell Lines and Plasmids

HeLa cells were grown at 37° C. in a 5% CO2 humid atmosphere in Dulbecco's Modified Eagle Medium (DMEM) supplemented with 4.5 g/L Glucose and 4.5 g/L Glutamine, 10% fetal bovine serum, 100 units/ml penicillin and 100 μg/ml streptomycin. Stable HeLa cell lines expressing H2B-mRFP/α-tubulin-GFP and H2B-mRFP/PA-α-tubulin-GFP (kind gift from P. Meraldi ETH, Zurich) were grown in presence of 400 μg/ml G418 and 20 μg/ml puromycin.

### Example 1.2. Plasmid and RNAi Transfection

To express fluorescently labelled proteins, cells were transfected for 48h using Lipofectamine 2000 (Invitrogen) with 1 μg of human TTLL11 tagged with GFP at either ends. hTTLL11, coming from hTTLL11 cloned in a peYFP-C1 vector (kind gift from C. Janke, Institut Curie, 91405 Orsay, France), was subcloned in peGFP-N1 and peGFP-C1 vectors (7).

To perform RNA interference (RNAi), HeLa cells were transfected at a 50-60% confluence using 500 pM RNAiMAX (invitrogen) with 100 nM siRNAs during 48h. siRNA smart pool (Dharmacon, SO-2796953G) was used with the following sequences:

SEQ ID NO: 1: TTLL11#1
5′-UGACGGAGAUGGUGCGUAA-3′;
SEQ ID NO: 2: TTLL11#2
5′-GAGUUUCAUUUCACGACAA-3′;
SEQ ID NO: 3: TLL11#3
5′-UCAAAUGGUGAAAGACGAU-3′;
SEQ ID NO: 4: TTLL11#4
5′-GGAUUCUGCCUGACGAGUU-3′;
SEQ ID NO: 5: Nuf2
5′-AAGCATGCCGTGAAACGTATA-3′.

### Example 1.3. SDS-PAGE and Western Blot Analysis

For western blot analysis the following primary antibodies were used: rabbit anti-polyE antibody (polyE, home-made, 1.1 μg/μl) diluted 1:1000, mouse anti-α-tubulin (DMIA, Sigma T9026) diluted 1:1000; rabbit anti-GFP (GFP, home-made, 0.6 μg/μl) diluted 1:500. Western blots were developed with Odyssey CLx imaging system (LI-COR Bioscience).

### Example 1.4. Immunofluorescence Microscopy

HeLa cells were grown on glass coverslip and fixed in ice-cold methanol for 3 min at −20° C. The following primary antibody were used: rabbit anti-polyE antibody (polyE, home-made, 1.1 μg/μl) diluted 1:1000, mouse anti-α-tubulin (DMIA, Sigma T9026) diluted 1:1000; rabbit anti-GFP (GFP, home-made, 0.6 μg/μl) diluted 1:1000; rabbit anti-β-tubulin (Abcam ab6046) diluted 1:500; mouse anti-glutamylated tubulin (GT335; Enzo 804-885) diluted 1:1000; human anti-centromere proteins (CREST, Antibodies Incorporated 15-235) diluted 1:100; anti-Hec1 (Hec1, Genentek GTX70268) diluted 1:100. DNA was counterstained with DAPI (1 μg/ml; Sigma-Aldrich) diluted 1:1000. Antibodies were diluted in the following buffer: PBS 1×, 0.1% Triton-X-100 (v/v), 0.5% BSA (w/v) Images were acquired with Leica DEI 6000B microscope mounted with a DF2 90000GT camera.

### Example 1.5. Quantification of Immunofluorescence Samples

The length of HeLa cells metaphase spindles was obtained by tracing manually a line from pole-to-pole using ImageJ. ImageJ scale was checked for correct pixel/μm conversion. To quantify the level of tubulin (poly) glutamylation in interphase and mitotic cells, the signal intensity for the selected antibody was normalized with the DMIA tubulin signal. To limit signal fluctuation the following formula was used:

$${NormalizedIntensity}_{x} = \frac{{RawIntensity}_{x} - ( Area * \overline{{Noise}_{x}} )}{{RawIntensity}_{tub} - ( Area * \overline{{Noise}_{tub}} )}$$

where x is the MT PTM of interest, Area is the circular ROI drawn around the metaphase spindle and must be identical for both signals, N̅o̅i̅s̅e̅ is the mean of the average intensity signal of 3 random areas within the cell around the spindle. The inter-kinetochore distance was obtained by tracing manually a straight line between sister chromatids centromeres detected with Hec1 (Hec1, Genentek GTX70268) antibody connected with CREST (CREST, Antibodies Incorporated 15-235) staining in metaphase spindles. Measurements were only validated for a given spindle when it was possible to obtain at least 5 different values. The lagging chromosome frequency was assessed in fixed HeLa cells stained with DAPI (1 μg/ml; Sigma-Aldrich) diluted 1:1000 to detect DNA and centromeres with an anti-CREST (CREST, Antibodies Incorporated 15-235) diluted 1:100. It was calculated by monitoring the presence of lagging chromosomes (showing both DNA and centromere positive signals) in between the two main mass of separating chromosomes in at least 121 anaphase cells. Data where then analysed with Prism6 (Graphpad).

### Example 1.6. K-Fibre Cold Stable Assay

Hela cells were cultured on 18 mm round coverslips in DMEM. Cells were washed 3 times during 5 min in PBS 1×. Medium was replaced by cold L15 medium at 4° C. and cells placed on ice. Coverslips were retrieved at given timepoints and cells fixed in ice-cold methanol at −20° C. during 3 min. Slides were stained for anti-α-tubulin (DM1A, Sigma T9026) diluted 1:1000 and DNA (DAPI 1 μg/ml; Sigma-Aldrich) diluted 1:1000. The quantification of K-fibre stability upon cell incubation on ice over time was obtained by scoring the status of the k-fiber microtubules among four arbitrarily defined classes.

### Example 1.7. Live Cell Imaging

HeLa cells stably expressing H2B-mRFP/α-tubulin-GFP were cultured in a 35/10 mm glass bottom, 4 compartment dish (Grainer Bio-one) and imaged using a 60× oil-immersion 1.4 NA objective on Andor Dragon Fly Spinning Disk confocal microscope. For imaging media was replaced with one without phenol red. Several random fields were selected for imaging to increase the possibility of visualizing mitotic events. Every field was imaged every 2-3 minutes during 6h taking a 15 μm Z-volume divided in 5-7 intervals depending on the experiment. Movies were then processed using ImageJ (ref. NIH Image to ImageJ: 25 years of image analysis). The STLC release experiment was performed by adding 10 μM STLC to growing HeLa cells for 2 h. The STLC was then washed out by 4 washes in warm PBS 1× and one with DMEM. Cells were placed under the microscope Andor Dragon Fly Spinning Disk microscope and imaged using a 60× oil-immersion 1.4 NA objective. Every field was imaged every 2-3 minutes during 6h taking a 15 μm Z-volume divided in 5-7 intervals depending on the experiment. The time required for cells to enter into anaphase was calculated from the time of the first wash. All displayed images represent maximum intensity projection of z-stacks. Andor Dragon Fly system was equipped with iXON-EMCCD Du-897 camera and Andor QI imaging software was used for images acquisition.

### Example 1.8. RT-qPCR HeLa Cells TTLLs Expression

For the RT-qPCR in HeLa cell, normal asynchronous Hela cell population was harvested, and the mRNA was isolated with TRIzol Reagent (Invitrogen). Total mRNA was quantified with a NanoDrop spectrophotometre and retro-transcribed in cDNA with the Superscript III (Invitrogen, 12574-026). cDNA was used for quantitative PCR with reverse transcription (RT-qPCR) analysis with SYBR green (ThermoFischer). Oligonucleotide sequence are indicated in Table S4.

TABLE S4
(SEQ ID NO: 6 to SEQ ID NO: 15)

| Target | sense | sequence |
|---|---|---|
| TTL11 | Fw | 5′-ACTTCTACCCTCGCTCATGG-3′ |
| | Rv | 5′-CCTGACAACCACCATCAGGTT-3′ |
| TTLL13 | Fw | 5′-ACCAACCCCTCTAACTCTTC-3′ |
| | Rv | 5′-TTTCCGCCTTCTCTTCCTC-3′ |
| Actin | Fw | 5′-CGAGAAGATGACCCAGATCATG-3′ |
| | Rv | 5′-CCACAGGACTCCATGCCCAGG-3′ |
| zfTTLL11 | Fw | 5′-GTGGACATCAAGAAGGTCTG-3′ |
| | Rv | 5′-AAAGTCTAGGACCCGAAAC-3′ |
| eef1a1 | Fw | 5′-GACATCCGTCGTGGTAATG-3′ |
| | Rv | 5′-GATGATGACCTGAGGTTG-3′ |

Table S4. Primers used for RT-qPCR

### Example 1.9. Tubulin Poleward Flux Measurement

HeLa cells stably expressing H2B-RFP/PA-α-tubulin-GFP were cultured in a 35/10 mm glass bottom, 4 compartments dish (Grainer Bio-one). For imaging cells were kept at 30° C. using an Okolab stage top chamber (UNO-T-H-CO2) and imaged using a 63× oil-immersion 1.4 NA objective lens on a Leica TCS SP5 confocal microscope. Images were acquired using the LAS X software. Bipolar spindles were identified by looking at the H2B-mRFP signal. PA-GFP-a-tubulin was activated in thin stripes (1-2 μm wide, as long as the metaphase plate) on one side of the metaphase plate with a 405 nm laser (100%). GFP fluorescence was captured every 8-10 s for 270 s. The poleward microtubule flux rate was calculated generating a kymograph of the fluorescent speckle (19) using ImageJ.

### Example 1.10. Zebrafish Husbandry, Strains

Zebrafish (Danio rerio) were maintained as previously described (W. Westerfield, The Zebrafish Book: A Guide for the Laboratory Use of Zebrafish (Brachydanio rerio) (Univ. of Oregon Press, 1995)). Wild-type embryos were obtained from the AB strain natural crosses and were kept in an incubator at 28° C. until sphere stage (according to Kimmel at al. Stages of embryonic development of the zebrafish. Dev Dyn 203:253-310, 1995). All protocols used for the experiments have previously been approved by the Institutional Animal Care and Use Ethic Committee (PRBB-IACUEC). The transgenic fish lines Tg(actb2:h2a-mCherry) (Zfin: e103Tg) was used for in vivo imaging experiments.

### Example 1.11. zfTTLL11 Cloning, RT-PCR and Mutagenesis

Total RNA was isolated from 24 hpf zebrafish embryos using tripleXtractor direct RNA (mirage biomedicals, GK23.0100) and reverse transcribed using a Xpert cDNA Synthesis kit (mirage biomedical GK80.0100).

zf_TTLL11 (ZDB-GENE-061013-747) cDNA was amplified using Phusion HF (Thermofisher F530S) and cloned into pCS2 vector (BamHI-EcoRI linearized) through Gibson cloning, using the following primers:

SEQ ID NO: 16:
TTLL11-pCS2-Fw:
5′-CTACTTGTTCTTTTTGCAGGATCCATGAGCGATCACTACGAGAGA
GT-3′
SEQ ID NO: 17:
TTLL11-pCS2-Rv:
5′-GCTCGAGAGGCCTTGAATTCTCAGTTGTCTGTGTTGGCTTTAGCA
G-3′

To visualize the protein, a Gibson system was used to subclone ttll11 into a pCS2 vector containing a GFP at the N-terminal end (XhoI linearized) to obtain the GFP-zfTTLL11 fusion protein. The following primers were used:

SEQ ID NO: 18: TTLL11-GFP-GibpCS2-Fw
CAAGGAATTCAAGGCCTCTCGAATGAGCGATCACTACGAGAGAGT
SEQ ID NO: 19:
TTLL11-GFP-GibpCS2-Rv
ACTCACTATAGTTCTAGAGGCTCAGTTGTCTGTGTTGGCT

For the semi-quantitative RT-PCR, zebrafish embryos at different stages of development were collected to extract mRNA with tripleXtractor direct RNA (mirage biomedicals, GK23.0100) and reverse transcribed using a Xpert cDNA Synthesis kit (mirage biomedical GK80.0100). 35 ng of cDNA were used for each stage of the RT-PCR reaction (Tm=57° C.; 33 cycles) and cDNA was amplified using GoTaq DNA polymerase (Promega, M7805). Oligonucleotide sequence are indicated in Table S4.

The pCS2-ttll11 construct was used to generate the mutated zfTTLL11.E466G version with QuickChange Site-Direct Mutagenesis kit (Agilent) with the following primers:

SEQ ID NO: 20: Fw: mutagenesis →
5′-CTTGAAGCCTGTTTTTATTAGGAGTCAATGCCAATCCCAGC-3′
SEQ ID NO: 21:
Rv: mutagenesis →
5′-GCTGGGATTGGCATTGACTCCTAATAAAACAGGCTTCAAG-3′

### Example 1.12. Morpholinos, mRNA Synthesis and Microinjection

Morpholino antisense oligonucleotides were designed and purchased from Gene Tools, LLC. To inhibit ttll11 we used a blocking translation SEQ ID NO: 22 MO (CGGCTGATTTGTTATCTCATCTAGG) and a standard control SEQ ID NO: 23 MO (CCTCTTACCTCAGTTACAATTTATA) as a negative control. We injected 2.8 ng of morpholino into one-cell stage embryos.

All capped mRNAs were synthesized using mMessage mMachine SP6 (Ambion, AM1340M). For the rescue experiments, 200 μg of ttll11 mRNA was injected together with the indicated MO. 200 μg of E466G mRNA (ttll11 mutated version) was injected together with the indicated MO as a negative control of the rescue experiments. GFP-Ttll11 mRNA was injected into one-cell stage embryos to visualize protein localization.

A PV820 microinjector (WPI) combined with a M3301R micro-manipulator was used to perform the microinjections.

### Example 1.13. Zebrafish Immunofluorescence

Zebrafish embryos at the sphere stage (2.5-4 hpf) were dechorionated and incubated overnight at RT shacking in the microtubule fixative solution (20). The MT-fixative was discarded, and the embryos were put in methanol at −20° C. overnight. After fixation was completed zebrafish embryos were transferred in a clean tube and washed 3 times during 5 min with tergitol at room temperature for rehydration. Embryos were then moved to a well of a 96-well plate and an overnight wash with the anti-autofluorescence buffer (PBS 1×, 100 mM NaBH₄) was performed at RT gently shacking. Buffer in the well was changed with TBS 1× for 5 extensive washes.

Embryos underwent the blocking step with TBS 1× with 2% BSA for 30 minutes RT gently shacking. Anti-polyE (1.1 μg/ml, home-made) and -β-tubulin (clone E7; Hybridoma Bank at University of Iowa) primary antibodies were added both at 1:200 dilution and left ON at 4° C. gently shacking. After primary antibody incubation 5 TBS 1× quick washes were performed and secondary antibodies Alexa Flour (Invitrogen) at 8 μg/ml were added for 3 hours at RT gently shacking. Embryos were then rinsed twice in TBS 1× and then left 20 minutes in DAPI (1 μg/ml; Sigma Aldrich) diluted 1:500, rinsed twice with TBS 1× and a last washed ON at 4° C. in TBS 1× before mounting. Embryos were then transferred in a tube containing low melting agarose at 42° C. diluted in TBS 1× and immediately placed in a Mattek dish with 7 mm diameter glass bottom and oriented with epithelial layer cells toward the glass slide before the agarose solidified. Once the agarose was stiff it was covered with TBS 1× to avoid evaporation.

### Example 1.14. Zebrafish Fixed Embryo Imaging

Fixed embryos were imaged in glass bottom Mattek dishes using a 63× oil-immersion 1.4 NA objective on a TCS SP5 inverted Leica confocal microscope. Laser intensity used changed through experiments according to the tubulin levels detected.

### Example 1.15. Zebrafish Embryo Live Imaging

For zfTTLL11 localization experiments zebrafish eggs were fertilized, collected and microinjected with zfTTLL11 mRNA with a micromanipulator (M3301R, WPI (World precision instruments). The quantity injected are relative to the ones specified in “Morpholinos, mRNA synthesis and microinjection” section, according to de experiment. Embryos at sphere stage were manually dechorionated and moved in a tube containing low melting agarose at 42° C. diluted in TBS 1× and immediately in a Mattek dish with 7 mm diameter glass bottom and oriented with epithelial layer cells toward the glass slide before the agarose solidified. Embryos were imaged under an Andor Revolution HD Spinning Disk microscope with a 60×, 1.4 NA oil objective with 2 min time-lapse intervals taking images every 2 μm in a 20 μm volume. iXON-EMCCD Du-897 camera and Andor IQ Imaging software was used for image acquisition. For anaphase lagging chromosome experiments zebrafish eggs coming from the Tg(bactin: H2AmCherry) were fertilized, collected and microinjected with either scramble or MO-1 (as in “Morpholinos, mRNA synthesis and microinjection”). Embryos were collected as above but laid on a 35/10 mm glass bottom, 4 compartment dish (Grainer Bio-one). Embryos were imaged using a Leica TCS SP8 confocal microscope equipped with an Argon laser. The objective used is an HC PLAN APO 63× 1.4 NA. Images were acquired simultaneously using the LAS X software. All the live imaging experiment were performed at RT.

### Example 1.16. Morphological Zebrafish Analysis

Zebrafish embryos at 36 hpf were anaesthetized with tricaine methane sulfonate (MS-222, Sigma Aldrich) The morphological phenotype was evaluated on site with an Olympus SZX16 scope equipped with an Olympus DP73 camera. Representative images were analyzed with ImageJ.

### Example 1.17. TCGA Data

The XenaBrowser (21) was used to obtain publicly available data on pan-cancer normalized gene expression, copy number variation, and somatic mutations in patients from The Cancer Genome Atlas (TCGA). The aneuploidy scores were obtained directly from Taylor et al. 2018 (18). Gene lengths were obtained from the latest human genome reference (GRCh38) made available through the object “ens.gene.ann.hg38” in the R package GeneBreak. From all cancer types available in TCGA, only those with at least 20 samples of solid tissue normal and primary tumor in the gene expression matrix (n=13) were considered. The full analysis pipeline is available at https://github.com/MiqG/Zadra_2021.

### Example 1.18. Differential Expression Analysis of TTLL11

A Wilcoxon Rank Sum test was performed for every type of cancer to assess statistically the differences in log-normalized read counts of TTLL11 between primary tumors and unmatched solid tissue normal samples.

### Example 1.19. Association Between TTLL11 Expression and Sample Aneuploidy

For every type of cancer, the Spearman correlation between log-normalized read counts of every gene and corresponding sample aneuploidy score was computed. Then, we assessed how likely it is to obtain the correlation coefficient of TTLL11 with respect to the rest of the genes. We standardized the correlation coefficients and computed the one-sided p-value of the correlation of TTLL11 with respect to the full distribution. Finally, to have an overview of the association between TTLL11 expression and aneuploidy across cancers with respect to the rest of the genes, we computed the median correlation coefficient across all cancer types and re-computed the p-value as explained.

### Example 1.20. Mutation Frequency of TTLL11

For every type of cancer and annotated mutation effect, we computed the mutation frequency per gene and divided it by the corresponding gene length to obtain the frequency of mutation per kilobase. Then, we followed the same procedure explained above to obtain the one-sided p-value of the standardized mutation frequency of TTLL11 per kilobase across cancer types and mutation effect with respect to the rest of the genes.

### Example 1.21. Sequence Sampling

Amino acid sequences were retrieved from ensembl (www.ensembl.org). Annotated canonical sequences of human (GRCh38.p13) TTLLs and their orthologous were used for the following species: Mus Musculus (GRCm39), Danio Rerio (GRCz11), Drosophila Melanogaster (BDGP6.32) and Caenorhabditis Elegans (WBcel235).

### Example 1.22. Alignment Estimation and Phylogeny Using Bali-phy

Popular alignment tools fail to provide a reliable alignment due to the highly divergent protein sequence employed in this analysis (22). Hence, Bali-phy v.3 (23) was implemented to analyze the TTLL aminoacidic dataset; this program uses a model-based approach, so the alignment is calculated alongside the phylogenetics analysis using a Bayesian inference. This method estimates the alignment with a likelihood averaged across several alignments (24). This multiple alignment approach is superior for estimating alignment in highly divergent sequences than the most common alignment tool as MAFT and MUSCLE (25, 26). Moreover, it overcomes the alignment-guided tree that relies already on a tree topology for estimating the alignment (22). The analysis was run tree times to check for convergence among different runs. Parameters used were-alphabet Amino-Acids-smodel gtr. Rates.gamma (25)-imodel rs07.

### Example 2. Results

### Example 2.1. TTLL11 as Therapeutic Target

Chromosome segregation fidelity is essential for the viability and genomic integrity of the daughter cells. It requires the correct bi-orientation of all chromosomes, a critical process monitored by the spindle assembly checkpoint (SAC), a surveillance mechanism that delays anaphase until all kinetochores pairs are correctly attached to microtubules (MTs) emanating from the opposite spindle poles (1). However, SAC-dependent arrest is not triggered by kinetochore-MT merotelic attachments that occur when a kinetochore attaches simultaneously to MTs emanating from both spindle poles, thus leading to chromosome segregation errors (2, 3). Different mechanisms can increase the frequency of merotelic attachments including the reduction in MT dynamics (1).

Tubulin post-translational modifications (PTMs) have emerged as regulators of MT functions. Although spindle MTs are modified with a variety of tubulin PTMs, little is known about the role these PTMs have in mitosis (2). So far, detyrosination was shown to guide metaphase chromosome congression through the motor protein CENP-E (3). By contrast, although there is an overall increase of polyglutamylase activity (4) and spindle MTs are specifically polyglutamylated (5) during mitosis the role of this PTM in cell division has not been unveiled. In fact, this is a highly complex PTM including different reactions and modified sites and the large number of enzymes involved.

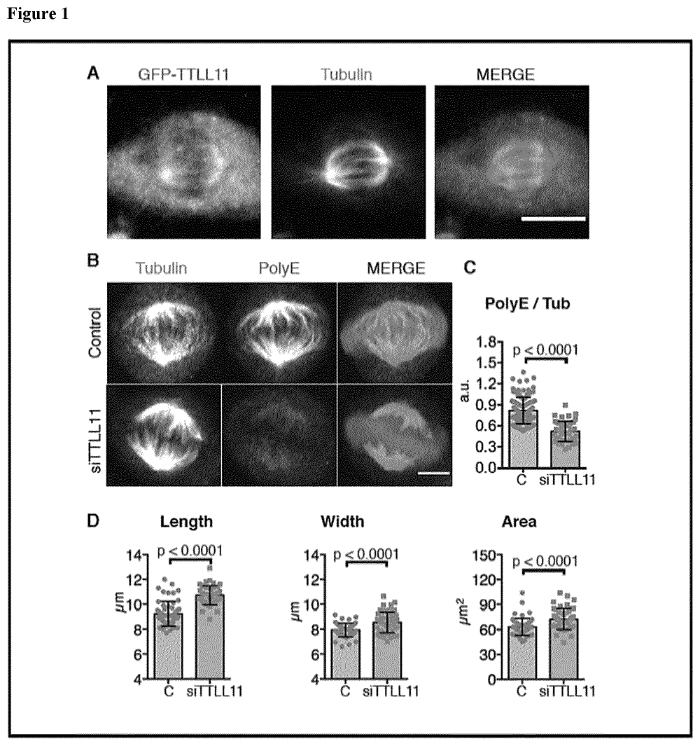

To address the molecular mechanisms and functional implications of the polyglutamylation of spindle MTs we aimed at identifying the enzyme driving this PTM. We first checked whether any of the 9 human TTLL glutamylase enzymes (Fig. S1) (6) localized to the spindle by expressing each protein with a fluorescent tag at their C or N terminus in Hela cells. Only TTLL11 (FIG. 1A) and TTLL13 (Fig. S2) localized to the spindle independently of the position of the fluorescent tag. The expression profiles for these two enzymes in human tissues showed that in contrast to TTLL13, TTLL11 is expressed in the majority of human tissues as well as in tissue culture cells such as HeLa cells (Fig. S3A-B), suggesting TTLL11 could be the main enzyme driving tubulin polyglutamylation in mitosis. To confirm this hypothesis, we silenced TTLL11 expression in Hela cells (Fig. S4A-B) and quantified the level of MT polyglutamylation in the spindle by immunofluorescence using two different antibodies: antibody GT335 that detects chains of one or more glutamates (Fig. S5) and PolyE that binds to glutamate chains of more than three residues (Fig. S5). While no significant differences were detected for GT335 (Fig. S6A-B), the signal for PolyE was significantly reduced in spindles assembled in siTTLL11 cells (0.52±0.15 a.u versus 0.82±0.19 a.u. in control; p<0.0001) (FIG. 1B) while no changes were found in the low signal detected in interphase cells (Fig. S7A-B). Consistently, Western blot analysis showed that the level of tubulin polyglutamylation in TTLL11-silenced cells was reduced in mitosis compared to controls (Fig. S8). Altogether, these results point at TTLL11 as the polyglutamylase that generates long glutamate chains on spindle MTs.

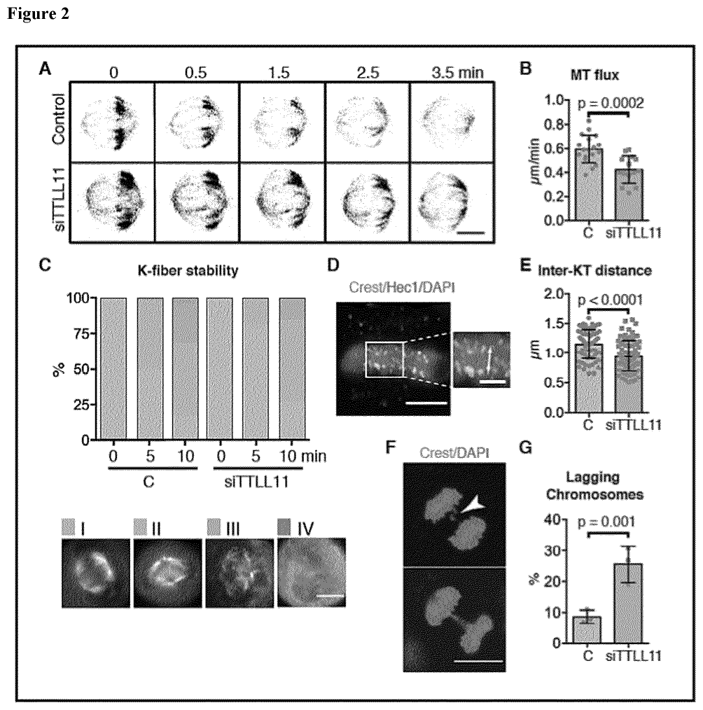

To investigate the functional consequences of MT polyglutamylation on spindle assembly and chromosome segregation, we used time-lapse imaging to monitor control and TTLL11-silenced HeLa cells undergoing mitosis. Silenced cells assembled bipolar spindles (Fig. S9A), segregated chromosomes without significant delays (Fig. S9B) and had a similar mitotic index (Fig. S9C). However, spindles assembled in TTLL11-silenced cells were overall 13% bigger than controls (10.72±0.15 μm versus 9.24=1.00 μm; p<0.0001) (FIG. 1D), suggesting reduced MT dynamics. The spindle flux was significantly reduced in TTLL11-silenced cells (0.42±0.11 μm/min versus 0.59±0.12 μm/min in control; p<0.0002) (FIG. 2A-B). Moreover, K-fibers were more resistant to cold-induced MT depolymerization in TTLL11-silenced cells indicating an increased stability (FIG. 2C, Table S1).

TABLE S1
Frequency (%)

| | | Time | | | | | |
|---|---|---|---|---|---|---|---|
| N = 3 | | (min) | n | I (%) | II (%) | III (%) | IV (%) |
| | Control | 0 | 40 | 100 | 0 | 0 | 0 |
| | Control | 5 | 29 | 41.00 | 53.9 | 5.1 | 0 |
| | Control | 10 | 43 | 9.3 | 41.9 | 41.9 | 7 |
| | siTTLL11 | 0 | 40 | 100 | 0 | 0 | 0 |
| | siTTLL11 | 5 | 57 | 18.9 | 211 | 0 | 0 |
| | siTTLL11 | 10 | 59 | 18.6 | 67.8 | 11.9 | 1.7 |

Table S1. K-fibre stability cumulative bar plot (FIG. 2B)

The inter-kinetochore distance in metaphase was smaller in the silenced cells (0.96±0.26 μm versus 1.16±0.25 μm in control; p<0.0001) suggesting a reduction of the inter-kinetochore tension (7) (FIG. 2D-E). Altogether these data pointed to reduced spindle MT dynamics in TTLL11-silenced cells that, however, did not prevent bipolar spindle assembly nor induced a mitotic delay. Previous studies showed that the partial stabilization of MTs can compromise chromosome segregation fidelity (8). We therefore monitored chromosome segregation fidelity and found a significant increase of lagging chromosomes in TTLL11-silenced anaphase cells (25.53±5.94% versus 8.6±2.21% in control; p=0.001) (FIG. 2F-G). Altogether, our data suggested that TTLL11-silenced cells enter anaphase with merotelic chromosomes that are not detected by the SAC and result in lagging chromosomes and mis-segregation.

We then directly tested whether siTTLL11 cells have an intact error correction mechanism (9). We used an Eg5 inhibitor (STLC) to induce monopolar spindles that accumulate a large number of erroneous chromosome attachments and followed mitotic progression upon drug washout by time lapse imaging. Control and siTTLL11 cells entered anaphase with similar kinetics, suggesting that the error correction mechanism is not compromised in the silenced cells (Fig. S10A-B). These results suggest that the increased stability of K-fibers in TTLL11-silenced cells are the direct cause of chromosome segregation errors due to the presence of unresolved merotelic attachments (7, 10). This indicates that chromosome segregation fidelity is not only secured by the SAC but also by the polyglutamylation of spindle MTs (and K-fibers) (11).

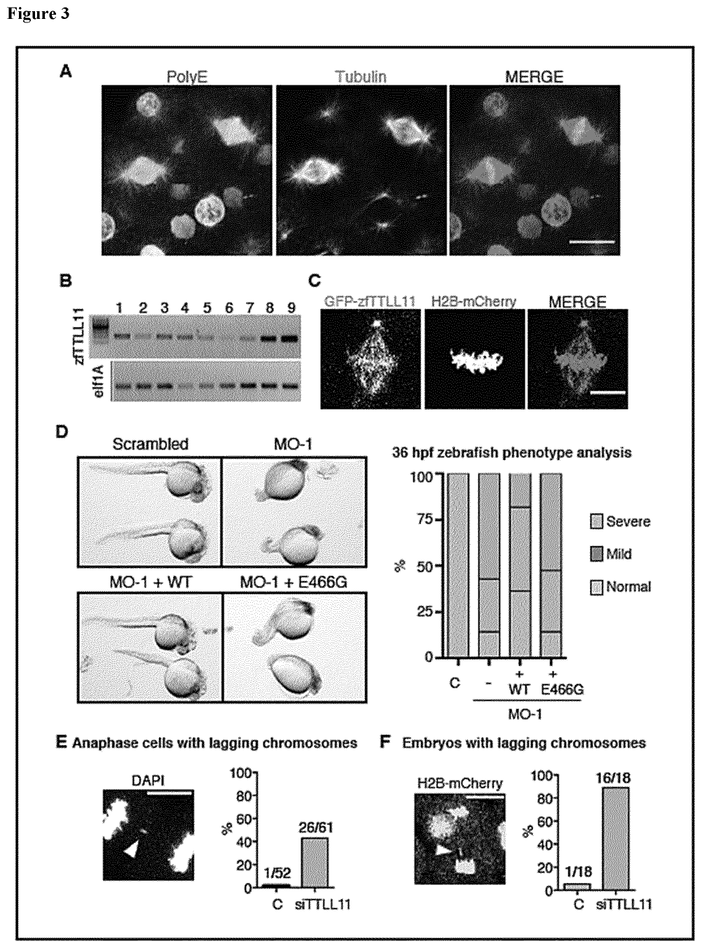

To address the relevance of this novel mechanism in vivo, we used zebrafish embryos (12). Immunofluorescence analysis using our PolyE antibody showed that spindle MTs are polyglutamylated in blastula stage embryos (4 hpf, hours post fertilization) (FIG. 3A). We also found that the highly conserved TTLL11 zebrafish ortholog (Fig. S1) is expressed in embryos from the 4-cell stage to 24 hpf (FIG. 3B). Moreover, time lapse imaging of blastula embryos (4 hpf) expressing GFP-zfTTLL11 showed that it localized to the spindle as observed in mammalian cells (FIG. 3C).

We then monitored the consequences of zfTTLL11 downregulation on early embryonic development that relies on multiple rounds of cell divisions. We found that 60% of the embryos injected with zfTTLL11-morpholino (zfTTLL11-MO) showed developmental defects ranging in severity from mild tilted tails to gross abnormalities (FIG. 3D, Table S2).

TABLE S2
Frequency (%)

| | | n | Normal (%) | Mild (%) | Severe (%) |
|---|---|---|---|---|---|
| N = 4 | Scramble | 31 | 100 | 0 | 0 |
| | MO-1 | 20 | 14.3 | 28.6 | 57.1 |
| | MO-1 + WT | 30 | 36.4 | 45.5 | 18.2 |
| | MO-1 + E466G | 20 | 14.3 | 33.3 | 52.4 |

Table S2. Zebrafish embryos phenotype analysis at 36 hpf (FIG. 3D).

There was also an increase of embryonic lethality of zfTTLL11-MO injected embryos (Fig. S11A). Co-injection of zfTTLL11-morpholinos and mRNA encoding GFP-zfTTLL11 rescued embryo early development and reduced the percentage of early embryonic death (FIG. 3D, Fig. S11A). We then tested whether the catalytic activity of zfTTLL11 is necessary and sufficient to support early embryonic development by co-injecting zfTTLL11-MO with mRNA encoding a catalytically inactive version of GFP-zfTTLL11 (point mutation E466G) (6). These embryos showed major developmental defects comparable to those observed in embryos only injected with zfTTLL11-MO (FIG. 3D), indicating that the catalytic activity of zfTTLL11 is essential. Altogether, our data suggest that TTLL11-dependent spindle MT polyglutamylation is essential for early zebrafish embryo development (FIG. 3). To directly assess the role of TTLL11 in cell division, we monitored chromosome segregation in blastula stage embryos by 4D time-lapse imaging. Most zfTTLL11-MO injected embryos showed at least one anaphase cell with a lagging chromosome (FIG. 3E), whereas this was rare in control embryos.

Consistently, quantifying anaphase cells with lagging chromosomes in fixed embryos confirmed that chromosome segregation fidelity is compromised in zfTTLL11-MO injected embryos (FIG. 3F). In addition, these embryos had many cells with micronuclei that are characteristic of mis-segregation events arising from lagging chromosomes (Fig. S11B-C) (13, 14).

Altogether, our data indicate that the polyglutamylation of spindle MTs by TTLL11 is essential to prevent cells entering anaphase in the presence of merotelic attachments, thus preventing aneuploidy.

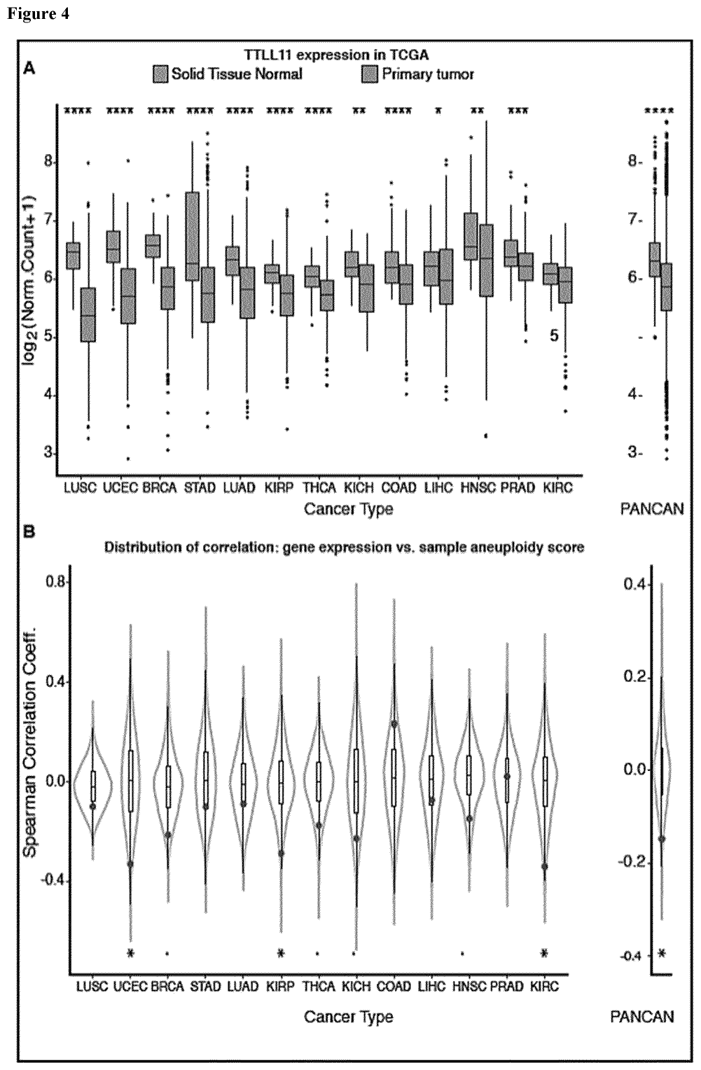

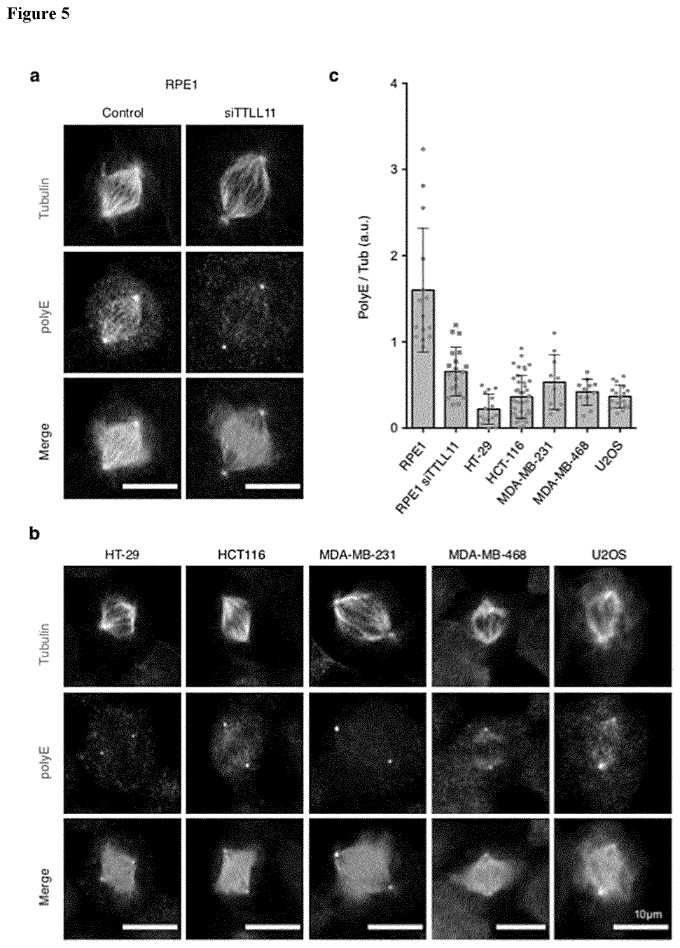

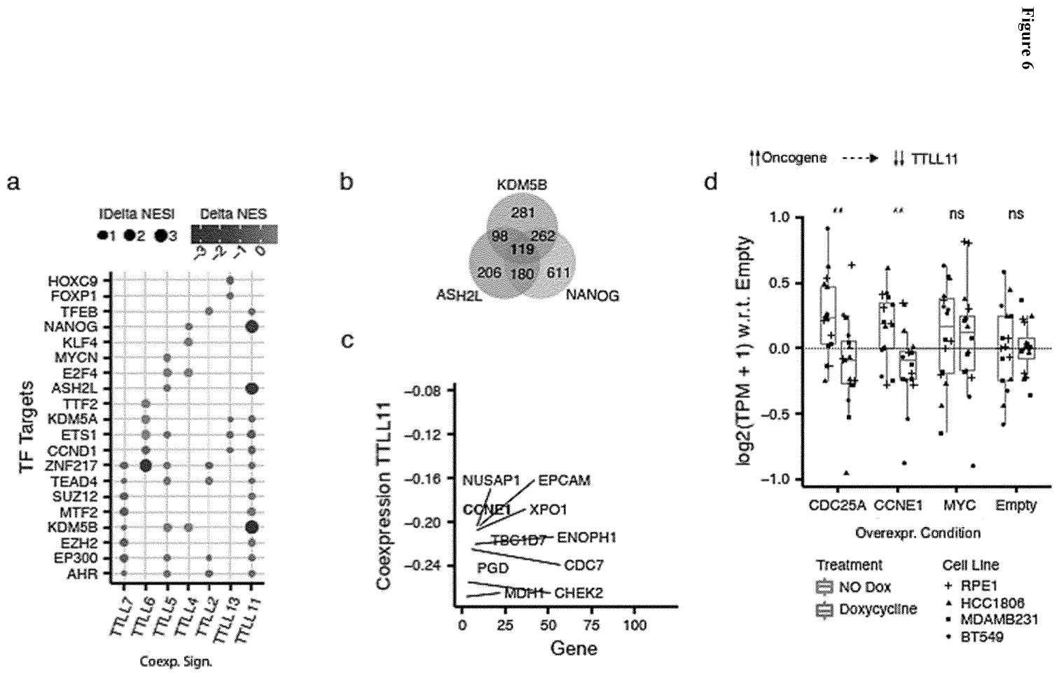

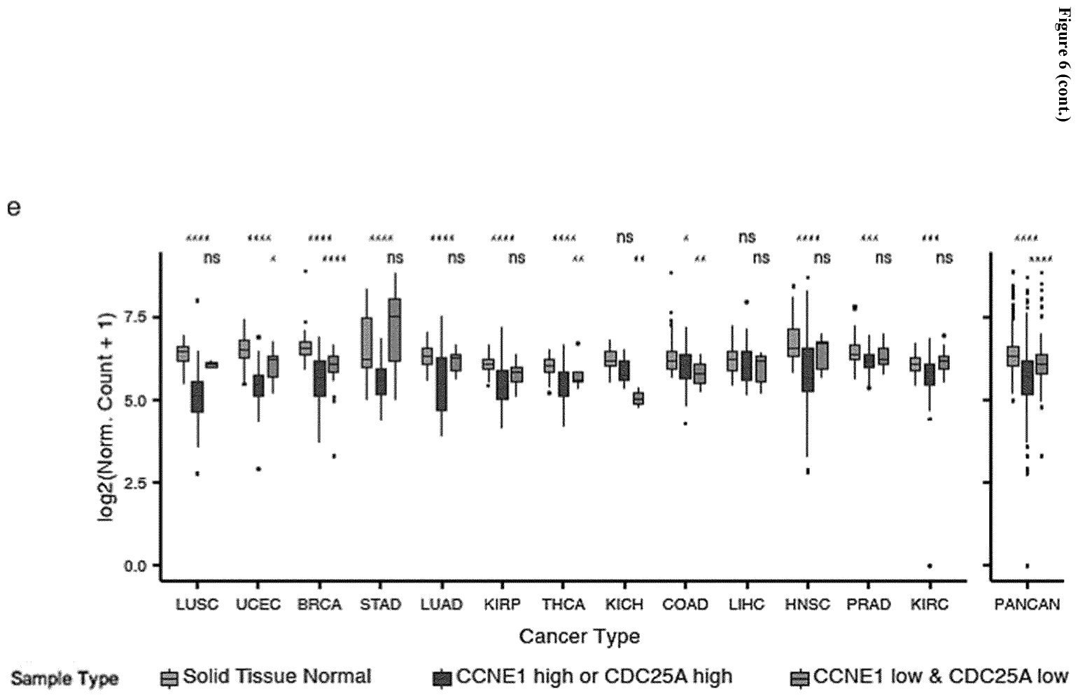

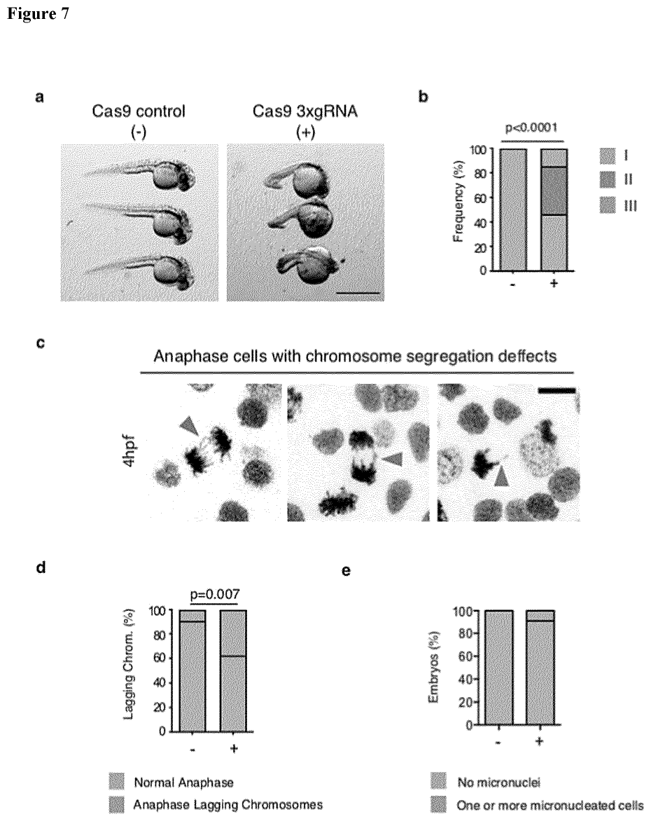

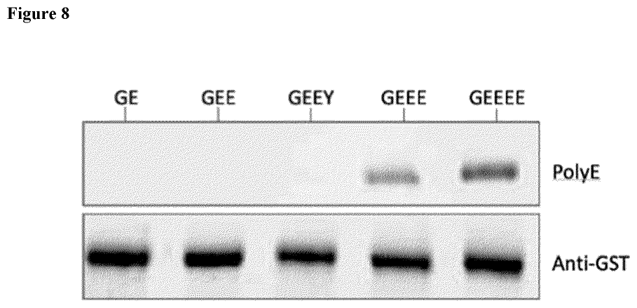

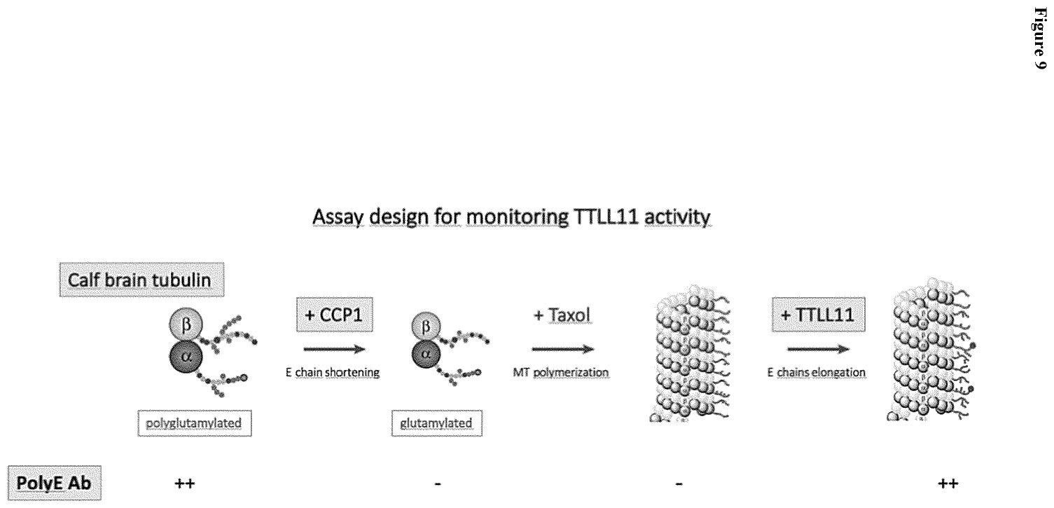

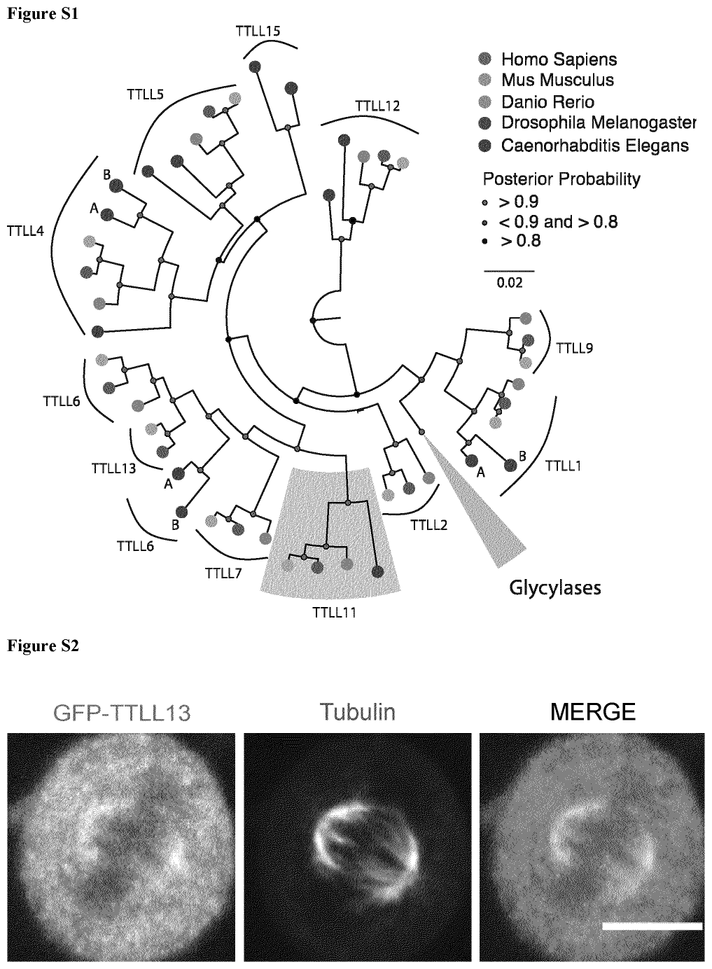

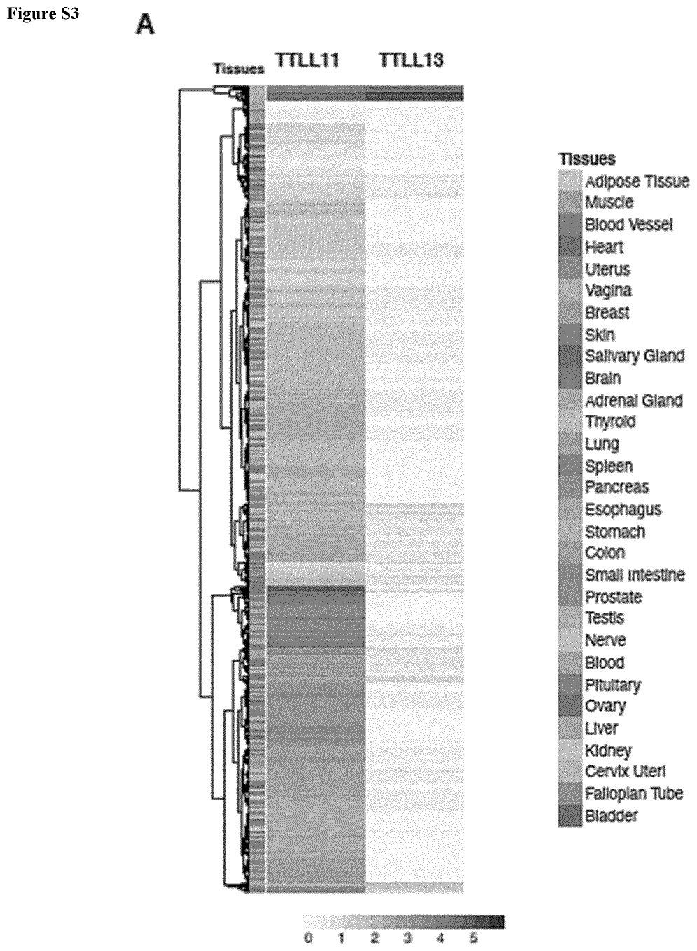

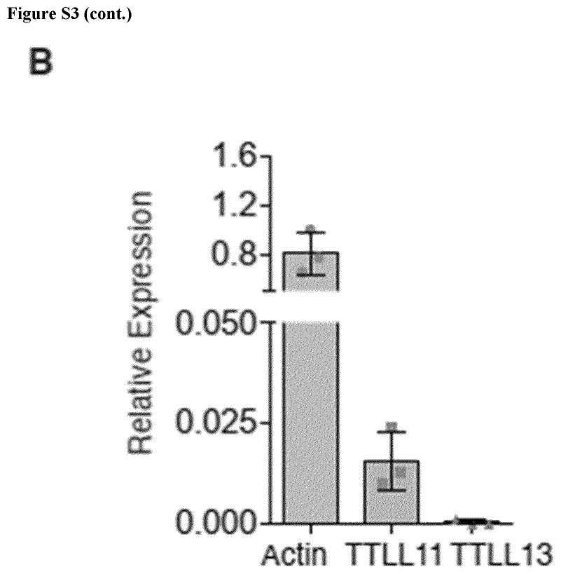

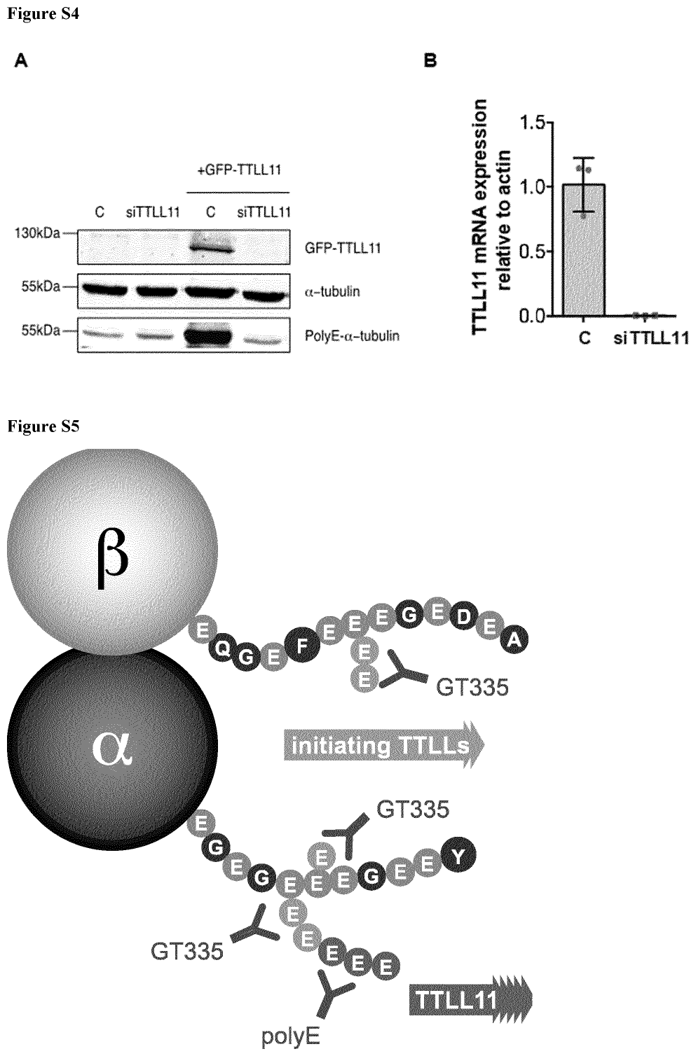

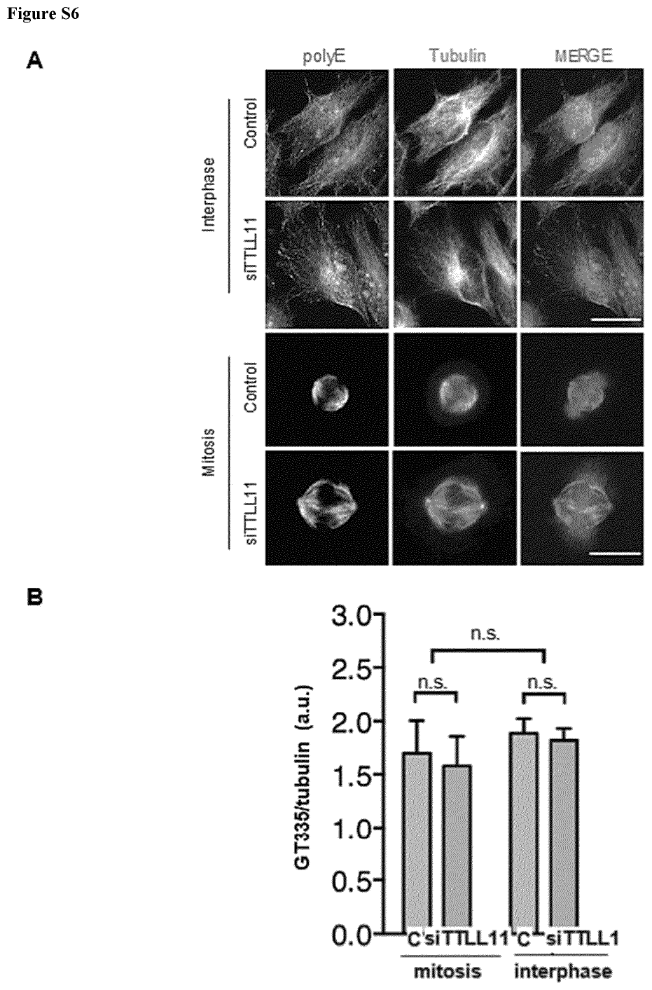

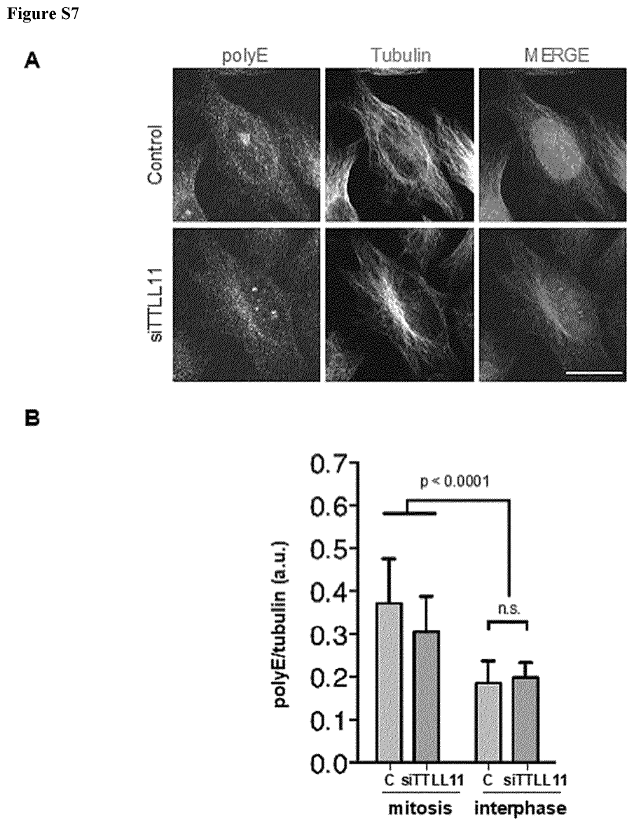

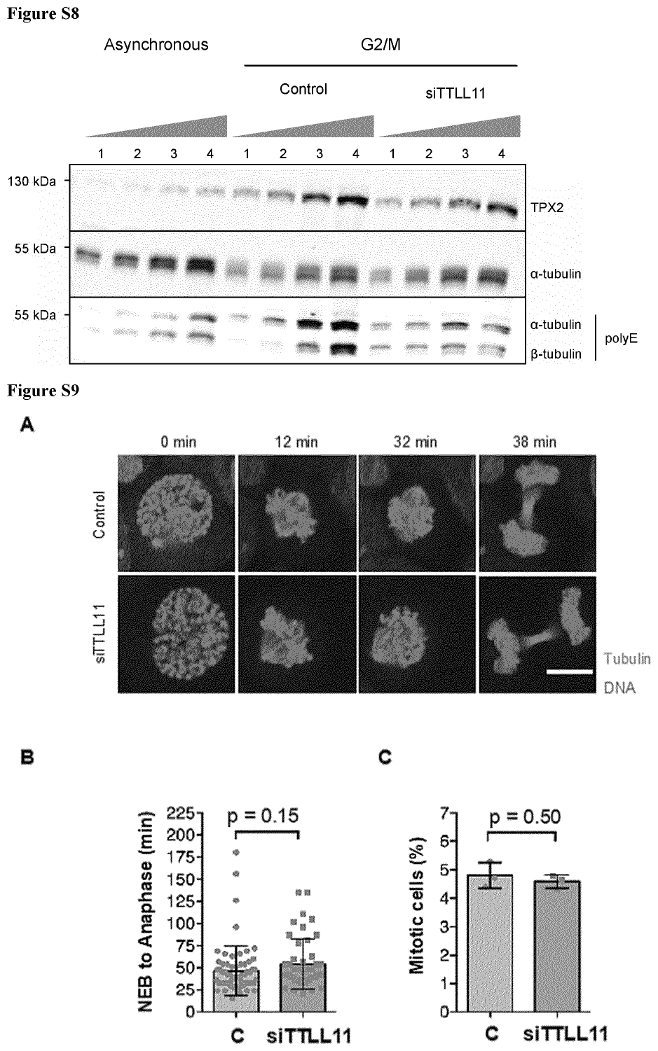

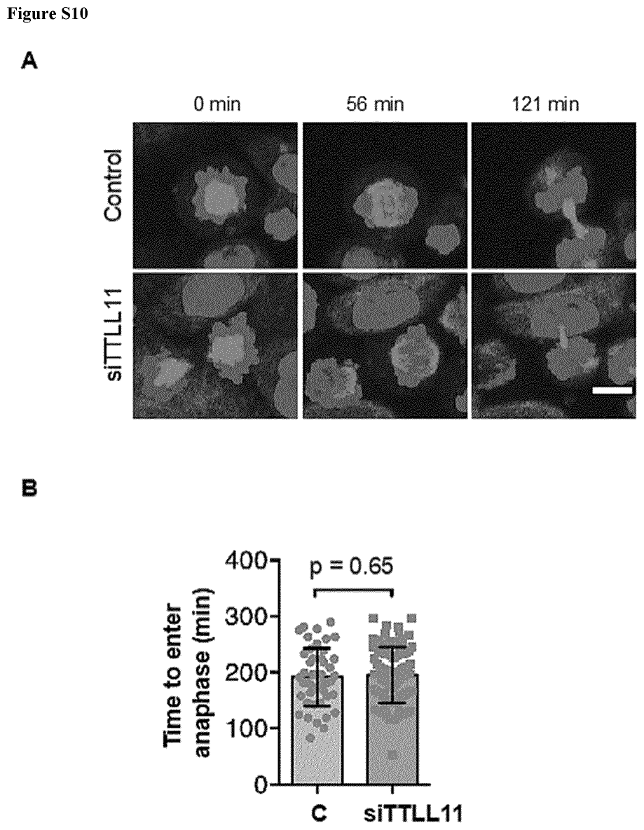

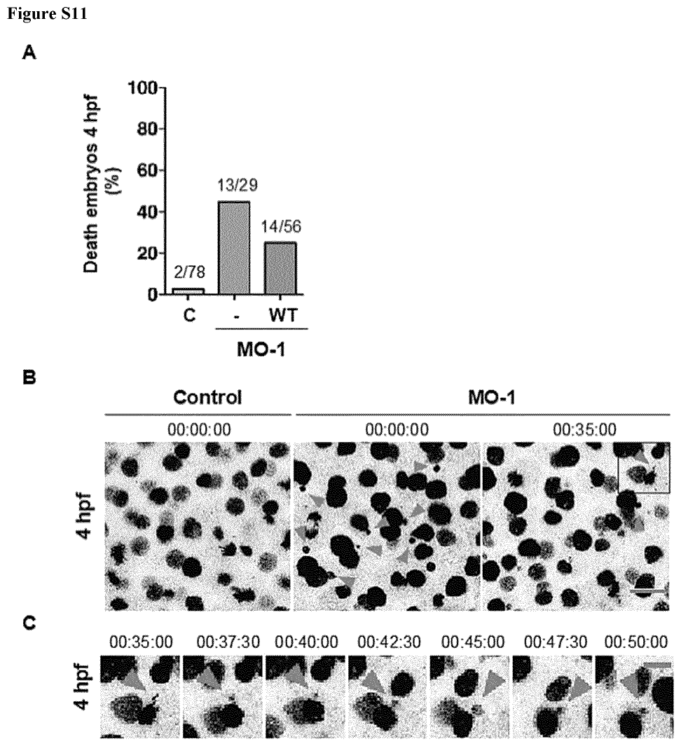

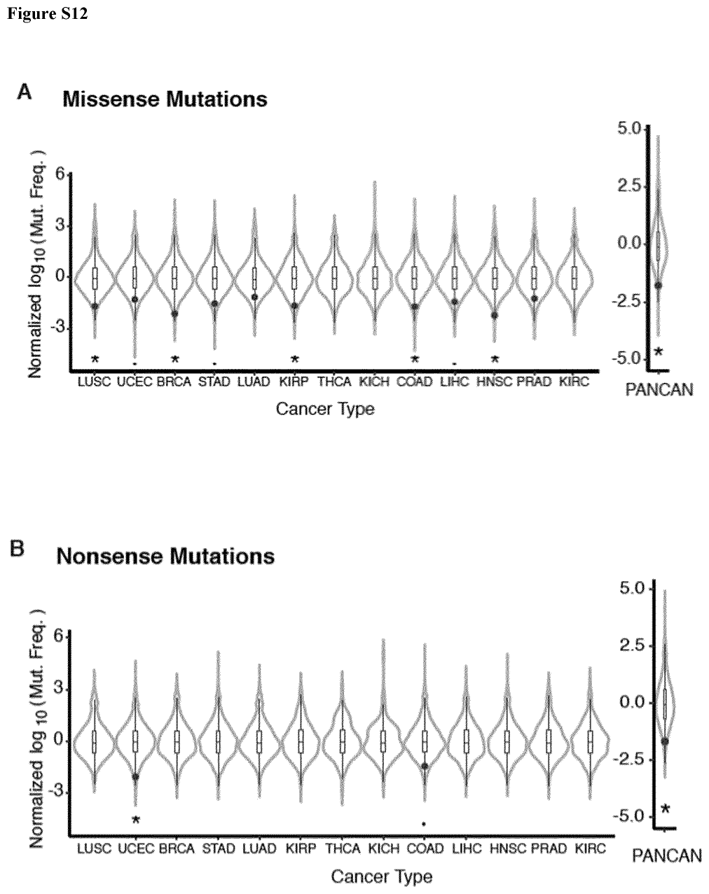

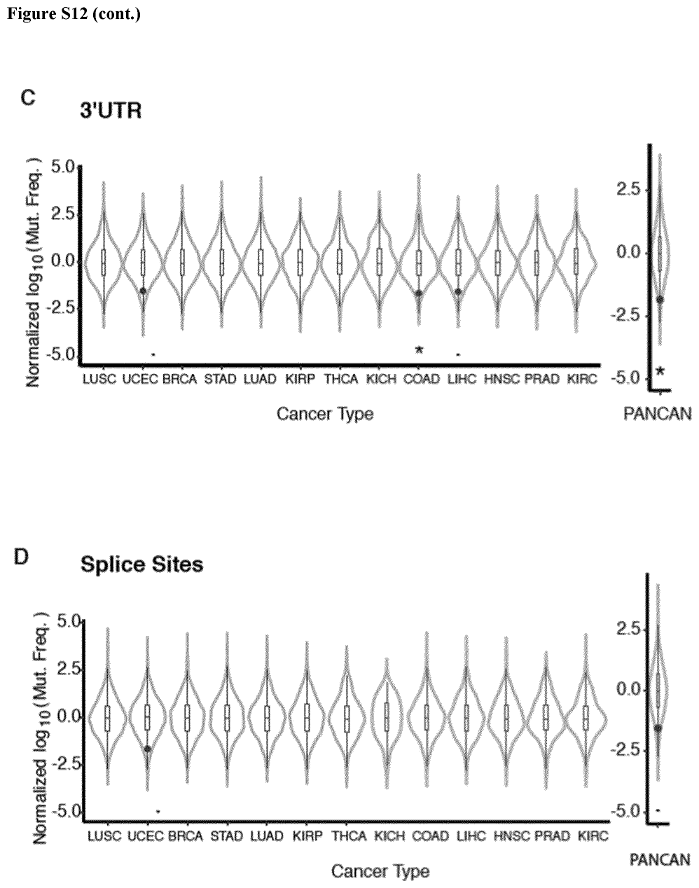

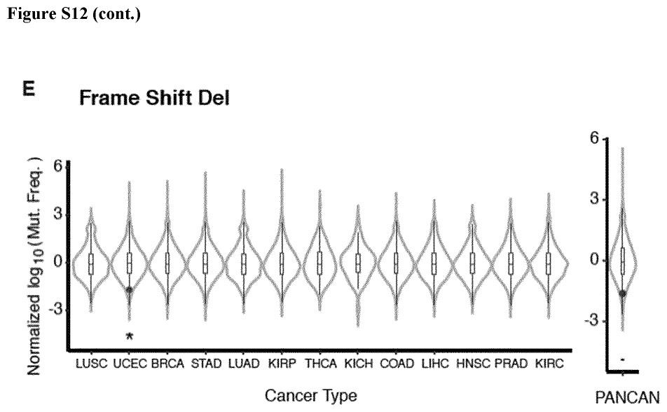

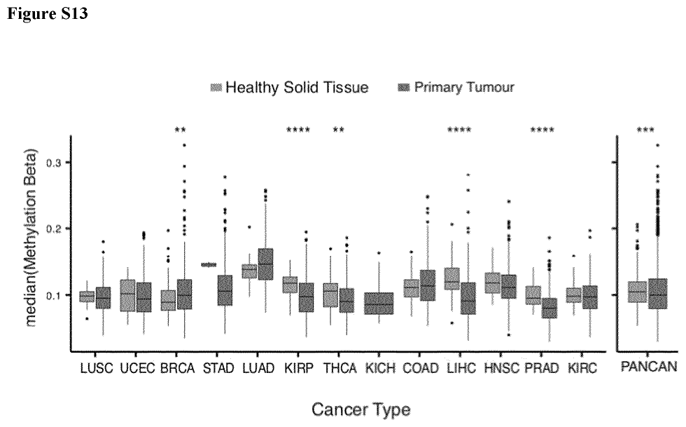

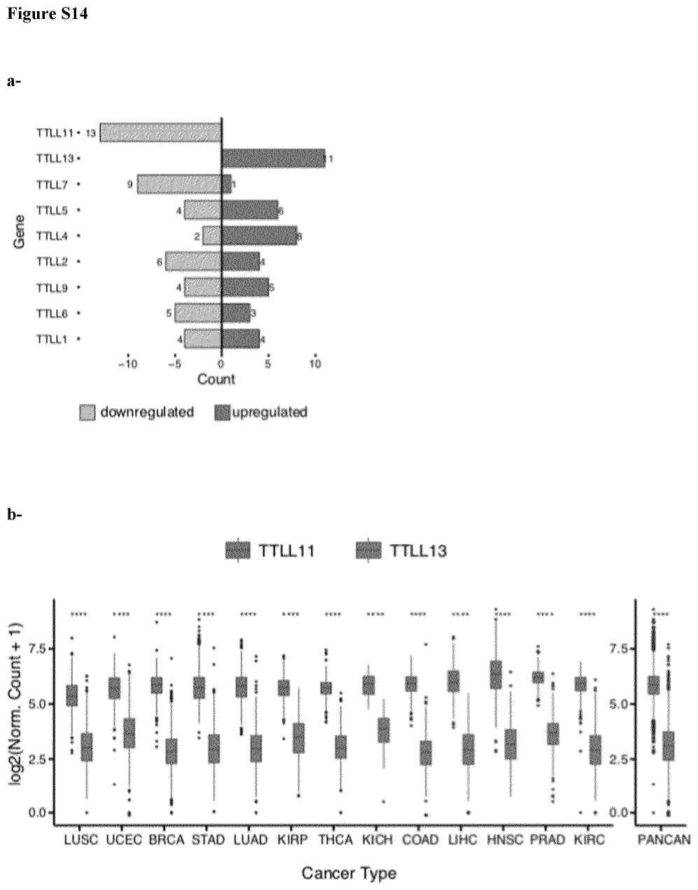

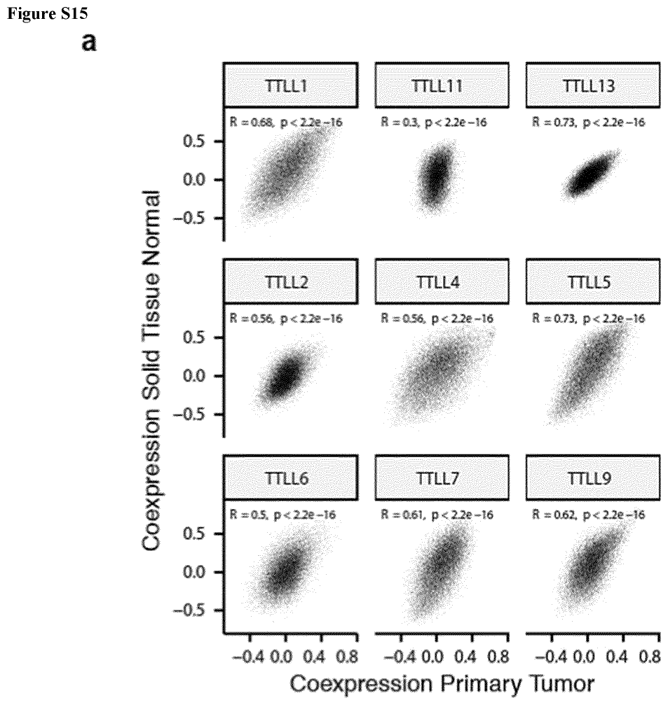

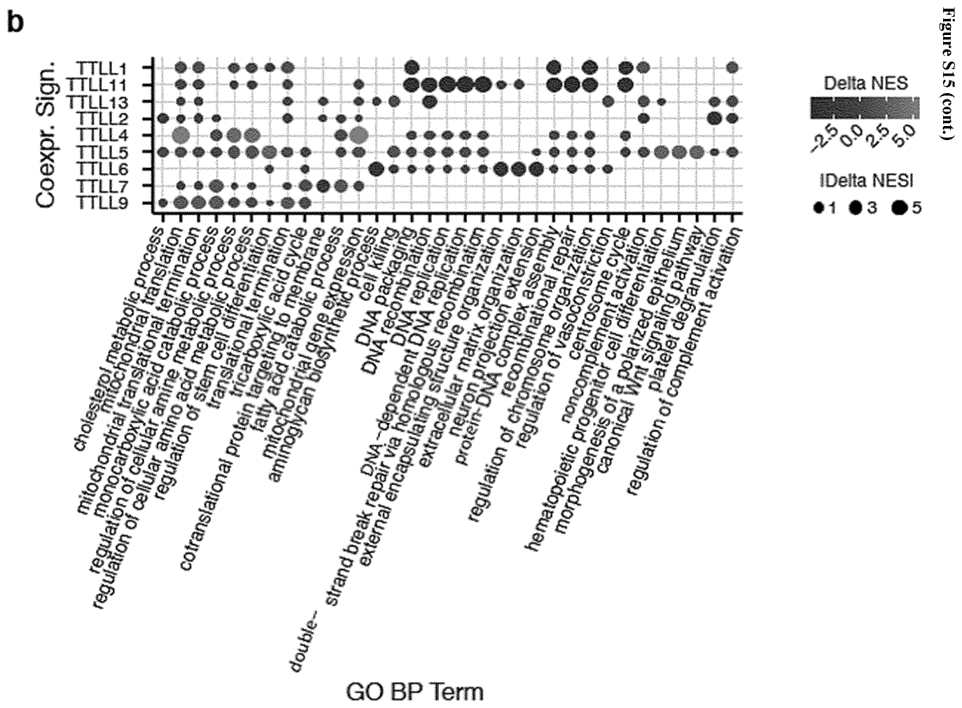

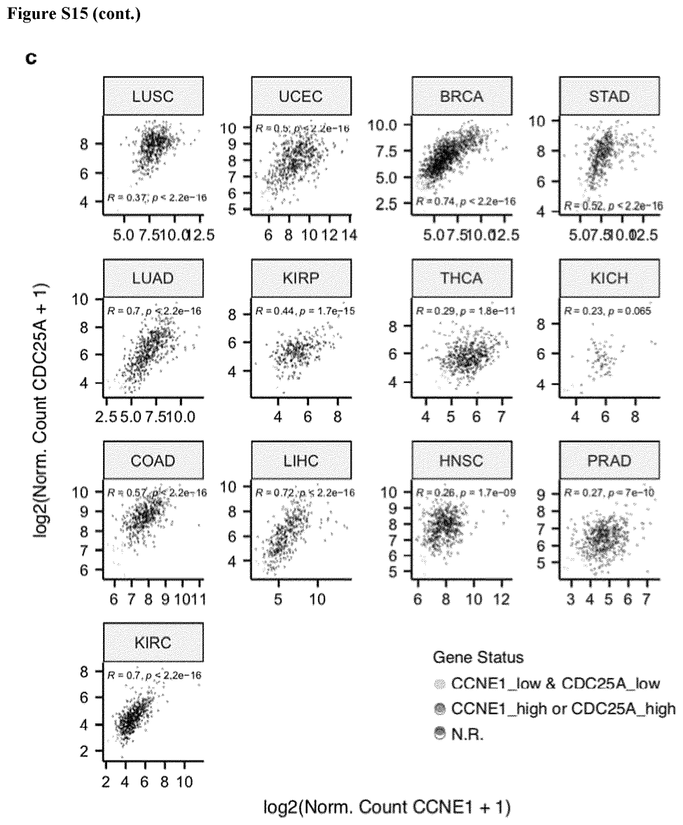

Aneuploidy is one of the most salient hallmarks of cancer. Approximately 86% of solid tumors are aneuploid (15) and many mis-segregate chromosomes at very high rates, a phenomenon called chromosomal instability (CIN). The most frequent cause of CIN in tumors is the presence of merotelic attachments (1, 16, 17) and cells with CIN were shown to have hyperstable k-MT attachments when compared to chromosomally stable diploid cells (1, 8, 10, 11). Altogether this suggested that mitotic MT polyglutamylation may be altered in cancer cells. We therefore analyzed whether TTLL11 expression or function may be altered in human tumors. Strikingly, we found that TTLL11 expression is significantly downregulated in all the tumors reported (FIG. 4A, Table S3).

TABLE S3
Log₂(Norm · Count + 1)

| cancer_type | sample_type | n | mean | SD |
|---|---|---|---|---|
| LUSC | Solid Tissue Normal | 51 | 6.375 | 0.311 |
| LUSC | Primary Tumor | 502 | 5.349 | 0.713 |
| UCEC | Solid Tissue Normal | 34 | 6.513 | 0.441 |
| UCEC | Primary Tumor | 532 | 5.672 | 0.701 |
| BRCA | Solid Tissue Normal | 114 | 6.580 | 0.356 |
| BRCA | Primary Tumor | 1097 | 5.816 | 0.555 |
| STAD | Solid Tissue Normal | 35 | 6.569 | 0.969 |
| STAD | Primary Tumor | 415 | 5.813 | 0.789 |
| LUAD | Solid Tissue Normal | 59 | 6.326 | 0.327 |
| LUAD | Primary Tumor | 515 | 5.745 | 0.751 |
| KIRP | Solid Tissue Normal | 32 | 6.079 | 0.304 |
| KIRP | Primary Tumor | 290 | 5.709 | 0.549 |
| THCA | Solid Tissue Normal | 59 | 6.005 | 0.278 |
| THCA | Primary Tumor | 505 | 5.714 | 0.427 |
| KICH | Solid Tissue Normal | 25 | 6.238 | 0.352 |
| KICH | Primary Tumor | 66 | 5.825 | 0.551 |
| COAD | Solid Tissue Normal | 41 | 6.365 | 0.633 |
| COAD | Primary Tumor | 452 | 5.902 | 0.491 |
| LIHC | Solid Tissue Normal | 50 | 6.221 | 0.443 |
| LIHC | Primary Tumor | 371 | 5.997 | 0.693 |
| HNSC | Solid Tissue Normal | 44 | 6.739 | 0.629 |
| HNSC | Primary Tumor | 520 | 6.339 | 0.953 |
| PRAD | Solid Tissue Normal | 52 | 6.468 | 0.448 |
| PRAD | Primary Tumor | 497 | 6.212 | 0.394 |
| KIRC | Solid Tissue Normal | 72 | 6.084 | 0.283 |
| KIRC | Primary Tumor | 533 | 5.863 | 0.566 |

Table S3. TTLL11 differential expression in tumors (FIG. 4A).

Moreover, there is a clear negative correlation between TTLL11 expression levels and aneuploidy (see Methods) (18) (FIG. 4B). Interestingly, the rate of missense mutations in the TTLL11 gene in cancer cells is significantly lower than expected (Fig. S12) suggesting that TTLL11 is essential for cell survival, and cancer cells lower its activity through the downregulation of its expression levels.

In summary, here we describe a previously unrecognized SAC-independent mechanism that ensures chromosome segregation fidelity during mitosis. This mechanism, based on the polyglutamylation of the spindle MTs by TTLL11, establishes and controls MT dynamics to ensure that cells do not enter anaphase in the presence of erroneous merotelic attachments. Moreover, we found that TTLL11 is consistently downregulated in most tumor cells, suggesting a novel mechanism that these cells may use to generate aneuploidies and favour CIN and cancer development.

### Example 2.2. Experimental Assays to Screen for Compounds with Inhibitory Activity Against TTLL11

Established assays to measure the activity of TTLL enzymes are based on measuring the incorporation of radioactive glutamate on taxol-stabilized microtubules prepared from purified brain tubulin upon incubation with the selected TTLL enzyme. This assay gives variable results and is very demanding technically. It is therefore only useful for testing a reduced number of conditions and it cannot be scaled up. Therefore, it cannot be adapted to medium or high throughput screening projects aiming at identifying compounds with inhibitory activity against a selected TTLL.

To overcome this limitation, we have developed non-radioactive assays for screening purposes. TTLL11 is an elongase that catalyses the addition of glutamates on a glutamate branching from the main C-terminal chain of tubulin (preferentially alpha or beta tubulin) on the surface of the microtubule. TTLL11 cannot catalyse the reaction that generates the first branching glutamate from the C-terminal chain of tubulin. Therefore, its substrate is a microtubule already modified with branching glutamates at the tubulin C-terminal chains.

To detect activity of TTLL11, a homemade polyclonal antibody (PolyE) that recognizes chains of at least three glutamates is used (see FIG. 8).

### Assay 1:

The first assay uses taxol stabilized microtubules prepared from purified calf brain tubulin. Tubulin can be purified from calf brains in the lab. This tubulin is highly modified and, in particular, it is polyglutamylated. To remove the long polyglutamate chains it can be incubated with carboxypeptidase CCP1 (expressed in human cells and purified by affinity chromatography in the lab). CCP1 is a deglutamylase that reduces the length of glutamate side-chains but does not eliminate the branched glutamate from the main tubulin C-terminal chain. The resulting ‘trimmed’ tubulin is then used to obtain taxol stabilized MTs that are excellent substrates for elongases such as TTLL11. The ‘trimmed’ MTs are then incubated either with lysates of human cells expressing recombinant TTLL11 (upon transfection of the corresponding constructs) or with purified enzyme (expressed in human cells or in baculovirus). A catalytic dead point mutant version of TTLL11 can be used as a control. The activity of the TTLL11 enzyme is then monitored by Western blot using the specific anti-PolyE antibody (FIG. 9). This assay can be adapted to an ELISA assay for medium or high throughput screenings. In this case taxol stabilized microtubules are prepared incorporating a little amount of rhodamine and biotin-labelled tubulin to allow respectively their visualization and potential immobilization at the bottom of a 96 well plate treated with PLL-PEG-biotin-neutravidin. They are then incubated with the purified TTLL11 enzyme and the selected compounds. The presence of polyglutamylated microtubules is detected using the polyE antibody by ELISA. The results are normalized by the total amount of microtubules in each well quantified by measuring rhodamine fluorescence or alternatively through an anti-tubulin antibody (DMIA). The specificity of the compound(s) with potential TTLL11 inhibitory activity can be tested using a similar assay with other selected TTLLs.

### Assay 2:

In this assay, peptides corresponding to the C-terminal region of tubulin (alpha or beta) will be synthetized. They will first be incubated with a TTLL enzyme (such as TTLL5) that can catalyze the branching of a glutamate from a glutamate present in the peptide. The modified peptides will then be applied to multiwell plates (96 or 384 wells). After incubation of the modified peptides with TTLL11 (as above), mass spectrometry will be used to determine whether additional glutamates have been incorporated to the peptides. This provides a direct read out of TTLL11 activity, allowing the high throughput screening of compound libraries.

### REFERENCES (MATERIAL AND METHODS)

1. L. M. Zasadil, E. M. Britigan, B. A. Weaver, 2n or not 2n: Aneuploidy, polyploidy and chromosomal instability in primary and tumor cells. Semin Cell Dev Biol 24, 370-379 (2013). doi: 10.1016/j.semcdb.2013.02.001
2. S. F. Bakhoum, G. Genovese, D. A. Compton, Deviant kinetochore microtubule dynamics underlie chromosomal instability. Curr Biol 19, 1937-1942 (2009). doi: 10.1016/j.cub.2009.09.055
3. M. Barisic et al., Mitosis. Microtubule detyrosination guides chromosomes during mitosis. Science 348, 799-803 (2015). doi: 10.1126/science.aaa5175
4. C. Janke, M. M. Magiera, The tubulin code and its role in controlling microtubule properties and functions. Nat Rev Mol Cell Biol 21, 307-326 (2020). doi: 10.1038/s41580-020-0214-3
5. C. Regnard, E. Desbruyeres, P. Denoulet, B. Edde, Tubulin polyglutamylase: isozymic variants and regulation during the cell cycle in Hela cells. J Cell Sci 112 (Pt 23), 4281-4289 (1999). doi: 10.1242/jcs.112.23.4281
6. B. Lacroix et al., Tubulin polyglutamylation stimulates spastin-mediated microtubule severing. J Cell Biol 189, 945-954 (2010). doi: 10.1083/jcb.201001024
7. J. van Dijk et al., A targeted multienzyme mechanism for selective microtubule polyglutamylation. Mol Cell 26, 437-448 (2007). doi: 10.1016/j.molcel.2007.04.012
8. D. Dudka et al., Complete microtubule-kinetochore occupancy favours the segregation of merotelic attachments. Nat Commun 9, 2042 (2018). doi: 10.1038/s41467-018-04427-x
9. K. M. Godek, L. Kabeche, D. A. Compton, Regulation of kinetochore-microtubule attachments through homeostatic control during mitosis. Nat Rev Mol Cell Biol 16, 57-64 (2015). doi: 10.1038/nrm3916
10. M. A. Lampson, K. Renduchitala, A. Khodjakov, T. M. Kapoor, Correcting improper chromosome-spindle attachments during cell division. Nat Cell Biol 6, 232-237 (2004). doi: 10.1038/ncb1102
11. S. F. Bakhoum, S. L. Thompson, A. L. Manning, D. A. Compton, Genome stability is ensured by temporal control of kinetochore-microtubule dynamics. Nat Cell Biol 11, 27-35 (2009). doi: 10.1038/ncb1809
12. S. L. Thompson, S. F. Bakhoum, D. A. Compton, Mechanisms of chromosomal instability. Curr Biol 20, R285-295 (2010). doi: 10.1016/j.cub.2010.01.034
13. D. A. Kane, C. B. Kimmel, The zebrafish midblastula transition. Development 119, 447-456 (1993).
14. D. Cimini, F. Degrassi, Aneuploidy: a matter of bad connections. Trends Cell Biol 15, 442-451 (2005). doi: 10.1016/j.tcb.2005.06.008
15. B. He et al., Chromosomes missegregated into micronuclei contribute to chromosomal instability by missegregating at the next division. Oncotarget 10, 2660-2674 (2019). doi: 10.1016/j.tcb.2005.06.008
16. S. L. Thompson, D. A. Compton, Examining the link between chromosomal instability and aneuploidy in human cells. J Cell Biol 180, 665-672 (2008). doi: 10.1083/jcb.200712029
17. K. W. Yuen, A. Desai, The wages of CIN. J Cell Biol 180, 661-663 (2008). doi: 10.1083/jcb.200801030
18. A. M. Taylor et al., Genomic and Functional Approaches to Understanding Cancer Aneuploidy. Cancer Cell 33, 676-689 e673 (2018). doi: 10.1016/j.ccell.2018.03.007
19. Maiato, Helder, ed. Cytoskeleton Dynamics: Methods and Protocols. Humana Press 15, 235-246 (2020). doi: 10.1007/978-1-0716-0219-5
20. Tran, L. D., Hino, H., Quach, H., Lim, S., Shindo, A., Mimori-Kiyosue, Y., . . . & Sampath, K. Dynamic microtubules at the vegetal cortex predict the embryonic axis in zebrafish. Development, 139, 3644-3652 (2012). doi: 10.1242/dev.082362
21. Goldman, M. J., Craft, B., Hastie, M., Repečka, K., McDade, F., Kamath, A., . . . & Haussler, D. Visualizing and interpreting cancer genomics data via the Xena platform. Nat Biotechnol 38, 675-678 (2020). doi: 10.1038/s41587-020-0546-8
22. Suchard, M. A.; Redelings, B. D. BAli-Phy: Simultaneous Bayesian inference of alignment and phylogeny. Bioinformatics, 22, (2006). 2047-2048 doi: 10.1093/bioinformatics/bt1175
23. Redelings, Benjamin D. BAli-Phy version 3: Model-based co-estimation of Alignment and Phylogeny. Bioinformatics 16, 2047-2048 (2020). doi: 10.1093/bioinformatics/btl175
24. Redelings, B. Erasing errors due to alignment ambiguity when estimating positive selection. Mol. Biol. Evol., 31, 1979-1993 (2014). doi: 10.1093/molbev/msu174
25. Katoh, K.; Rozewicki, J.; Yamada, K.D. MAFFT online service: Multiple sequence alignment, interactive sequence choice and visualization. Brief. Bioinform., 20, 1160-1166 (2018). doi: 10.1093/bib/bbx108
26. Edgar, R.C. MUSCLE: Multiple sequence alignment with high accuracy and high throughput. Nucleic Acids Res., 32, 1792-1797 (2004). doi: 10.1093/nar/gkh340
27. Garnham et al., Multivalent Microtubule Recognition by Tubulin Tyrosine Ligase-Like Family Glutamylases 161, 1112-1123 (2015). doi: 10.1016/j.cell.2015.04.003
28. Szyk et al., Tubulin tyrosine ligase structure reveals adaptation of an ancient fold to bind and modify tubulin. Nat Struct Mol Biol 18, 1250-1258 (2011). doi: 10.1038/nsmb.2148
29. Wolff, A., de Néchaud, B., Chillet, D., Mazarguil, H., Desbruyères, E., Audebert, S., . . . & Denoulet, P. Distribution of glutamylated alpha and beta-tubulin in mouse tissues using a specific monoclonal antibody, GT335. European journal of cell biology, 59, 425-432 (1992).
30. Rogowski, K., Van Dijk, J., Magiera, M. M., Bosc, C., Deloulme, J. C., Bosson, A., . . . & Janke, C. A family of protein-deglutamylating enzymes associated with neurodegeneration. Cell, 143, 564-578 (2010). doi: 10.1016/j.cell.2010.10.014

### REFERENCES (RESULTS)

1. L. M. Zasadil, E. M. Britigan, B. A. Weaver, 2n or not 2n: Aneuploidy, polyploidy and chromosomal instability in primary and tumor cells. Semin Cell Dev Biol 24, 370-379 (2013).
2. S. F. Bakhoum, G. Genovese, D. A. Compton, Deviant kinetochore microtubule dynamics underlie chromosomal instability. Curr Biol 19, 1937-1942 (2009).
3. M. Barisic et al., Mitosis. Microtubule detyrosination guides chromosomes during mitosis. Science 348, 799-803 (2015).
4. C. Janke, M. M. Magiera, The tubulin code and its role in controlling microtubule properties and functions. Nat Rev Mol Cell Biol 21, 307-326 (2020).
5. C. Regnard, E. Desbruyeres, P. Denoulet, B. Edde, Tubulin polyglutamylase: isozymic variants and regulation during the cell cycle in HeLa cells. J Cell Sci 112 (Pt 23), 4281-4289 (1999).
6. B. Lacroix et al., Tubulin polyglutamylation stimulates spastin-mediated microtubule severing. J Cell Biol 189, 945-954 (2010).
7. J. van Dijk et al., A targeted multienzyme mechanism for selective microtubule polyglutamylation. Mol Cell 26, 437-448 (2007).
8. D. Dudka et al., Complete microtubule-kinetochore occupancy favours the segregation of merotelic attachments. Nat Commun 9, 2042 (2018).
9. K. M. Godek, L. Kabeche, D. A. Compton, Regulation of kinetochore-microtubule attachments through homeostatic control during mitosis. Nat Rev Mol Cell Biol 16, 57-64 (2015).
10. M. A. Lampson, K. Renduchitala, A. Khodjakov, T. M. Kapoor, Correcting improper chromosome-spindle attachments during cell division. Nat Cell Biol 6, 232-237 (2004).
11. S. F. Bakhoum, S. L. Thompson, A. L. Manning, D. A. Compton, Genome stability is ensured by temporal control of kinetochore-microtubule dynamics. Nat Cell Biol 11, 27-35 (2009).
12. S. L. Thompson, S. F. Bakhoum, D. A. Compton, Mechanisms of chromosomal instability. Curr Biol 20, R285-295 (2010).
13. D. A. Kane, C. B. Kimmel, The zebrafish midblastula transition. Development 119, 447-456 (1993).
14. D. Cimini, F. Degrassi, Aneuploidy: a matter of bad connections. Trends Cell Biol 15, 442-451 (2005).
15. B. He et al., Chromosomes missegregated into micronuclei contribute to chromosomal instability by missegregating at the next division. Oncotarget 10, 2660-2674 (2019).
16. S. L. Thompson, D. A. Compton, Examining the link between chromosomal instability and aneuploidy in human cells. J Cell Biol 180, 665-672 (2008).
17. K. W. Yuen, A. Desai, The wages of CIN. J Cell Biol 180, 661-663 (2008).
18. A. M. Taylor et al., Genomic and Functional Approaches to Understanding Cancer Aneuploidy. Cancer Cell 33, 676-689 e673 (2018).

1. In vitro method for screening, identifying and/or producing compounds for treating a disease which comprises: a) Assessing TTLL11 enzyme activity once the candidate compound has been incubated with TTLL11 enzyme, and b) wherein if an inhibition of TTLL11 activity is observed, it is indicative that the candidate compound may be effective for treating a disease.

2. In vitro method, according to claim 1, characterized in that the method is a non-radioactive assay wherein TTLL11 activity is assessed by analysing whether additional glutamates haven been incorporated by the enzyme TTLL11 to the enzyme substrate, wherein if no additional glutamates have been incorporated to the enzyme substrate this is an indication that the candidate compound has inhibited TTLL11 activity and may be effective as for treating a disease.

3. In vitro method, according to claim 1, characterized in that TTLL11 activity is assessed by analysing whether additional glutamates haven been incorporated by the enzyme TTLL11 to the enzyme substrate by using an immunoassay or by mass spectrometry, wherein if no additional glutamates have been incorporated to the enzyme substrate this is an indication that the candidate compound has inhibited TTLL11 activity and may be effective for treating a disease.

4. In vitro method, according to claim 1, characterized in that the enzyme substrate is a stabilized microtubule which has been obtained by tubulin polymerization and the activity of TTLL11 enzyme is assessed by analysing whether additional glutamates haven been incorporated by the enzyme TTLL11 to the enzyme substrate by using an immunoassay.

5. In vitro method, according to claim 1, characterized in that the enzyme substrate is a peptide corresponding to the C-terminal region of tubulin and the activity of TTLL11 enzyme is assessed by analysing whether additional glutamates haven been incorporated by the enzyme TTLL11 to the enzyme substrate by mass spectrometry.

6. In vitro method, according to claim 1, for screening, identifying and/or producing compounds for the treatment of diseases benefiting from the inhibition of microtubule polyglutamylation in mitosis.

7. In vitro method, according to claim 1, for screening, identifying and/or producing compounds for the treatment of cancer.

8. A method of treating a disease with inhibitors of microtubule polyglutamylation in mitosis.

9. A method of treating disease with inhibitors of microtubule polyglutamylation in mitosis, according to claim 8, wherein the inhibitors are TTLL11 inhibitors.

10. A method of treating disease with inhibitors of microtubule polyglutamylation in mitosis, according to claim 8, in wherein the disease is a disease benefiting from the inhibition of microtubule polyglutamylation in mitosis.

11. A method of treating disease with inhibitors of microtubule polyglutamylation in mitosis, according to claim 8, wherein the disease is cancer.

12. A method of treating disease with inhibitors of microtubule polyglutamylation in mitosis, according to claim 8, wherein the inhibitor is a biologic agent or a chemical compound.

13. A method of treating disease with inhibitors of microtubule polyglutamylation in mitosis, according to claim 8, wherein the inhibitor is a small molecule.

14. A method of treating disease with inhibitors of microtubule polyglutamylation in mitosis, according to claim 8, wherein the inhibitor is a siRNA.

15. A method of treating disease with inhibitors of microtubule polyglutamylation in mitosis, according to claim 8, wherein the inhibitor is an ATP-competitive inhibitor.

16. Pharmaceutical composition comprising an inhibitor of microtubule polyglutamylation in mitosis and, optionally, pharmaceutically acceptable carriers or excipients.

17. Pharmaceutical composition, according to claim 16, comprising:
(a) a TTLL11 inhibitor and, optionally, pharmaceutically acceptable carriers or excipients;
(b) an inhibitor selected from a biologic agent or a chemical compound and, optionally, pharmaceutically acceptable carriers or excipients;
(c) a small molecule inhibitor and, optionally, pharmaceutically acceptable carriers or excipients;
(d) comprising a siRNA and, optionally, pharmaceutically acceptable carriers or excipients; or
(e) an ATP-competitive inhibitor and optionally, pharmaceutically acceptable carriers or excipients.

18. (canceled)

19. (canceled)

20. (canceled)

21. (canceled)

22. In vitro method for the diagnosis and/or prognosis of a disease benefiting from the inhibition of microtubule polyglutamylation in mitosis which comprises assessing the level of expression of TTLL11 in a biological sample obtained from the patient, wherein the identification of a reduced level of expression of TTLL11, as compared with a pre-established level of expression measured in healthy control patients, is as indication that the patient is suffering from a disease benefiting from the inhibition of microtubule polyglutamylation in mitosis.

23. In vitro method, according to claim 22, for the diagnosis and/or prognosis of cancer which comprises assessing the level of expression of TTLL11 in a biological sample obtained from the patient, wherein the identification of a reduced level of expression of TTLL11, as compared with a pre-established level of expression measured in healthy control patients, is as indication that the patient is suffering from cancer.

24. In vitro use of TTLL11 for the diagnosis and/or prognosis of a disease benefiting from the inhibition of microtubule polyglutamylation in mitosis.

25. In vitro use of TTLL11, according to claim 24, for the diagnosis and/or prognosis of cancer.


SEQUENCE LISTING (23 sequences)

SEQ ID NO: 1 - RNA, 19 nt, synthetic construct
  source 1..19: mol_type = other RNA
tgacggagatggtgcgtaa

SEQ ID NO: 2 - RNA, 19 nt, synthetic construct
  source 1..19: mol_type = other RNA
gagtttcatttcacgacaa

SEQ ID NO: 3 - RNA, 19 nt, synthetic construct
  source 1..19: mol_type = other RNA
tcaaatggtgaaagacgat

SEQ ID NO: 4 - RNA, 19 nt, synthetic construct
  source 1..19: mol_type = other RNA
ggattctgcctgacgagtt

SEQ ID NO: 5 - DNA, 21 bp, synthetic construct
  source 1..21: mol_type = other DNA
aagcatgccgtgaaacgtata

SEQ ID NO: 6 - DNA, 20 bp, synthetic construct
  source 1..20: mol_type = other DNA
acttctaccctcgctcatgg

SEQ ID NO: 7 - DNA, 22 bp, synthetic construct
  source 1..22: mol_type = other DNA
cctgacaaccaccattcaggtt

SEQ ID NO: 8 - DNA, 20 bp, synthetic construct
  source 1..20: mol_type = other DNA
accaacccctctaactcttc

SEQ ID NO: 9 - DNA, 19 bp, synthetic construct
  source 1..19: mol_type = other DNA
tttccgccttctcttcctc

SEQ ID NO: 10 - DNA, 22 bp, synthetic construct
  source 1..22: mol_type = other DNA
cgagaagatgacccagatcatg

SEQ ID NO: 11 - DNA, 21 bp, synthetic construct
  source 1..21: mol_type = other DNA
ccacaggactccatgcccagg

SEQ ID NO: 12 - DNA, 20 bp, synthetic construct
  source 1..20: mol_type = other DNA
gtggacatcaagaaggtctg

SEQ ID NO: 13 - DNA, 19 bp, synthetic construct
  source 1..19: mol_type = other DNA
aaagtctaggacccgaaac

SEQ ID NO: 14 - DNA, 19 bp, synthetic construct
  source 1..19: mol_type = other DNA
gacatccgtcgtggtaatg

SEQ ID NO: 15 - DNA, 19 bp, synthetic construct
  source 1..19: mol_type = other DNA
gatgatgacctgagcgttg

SEQ ID NO: 16 - DNA, 47 bp, synthetic construct
  source 1..47: mol_type = other DNA
ctacttgttctttttgcaggatccatgagcgatcactacgagagagt

SEQ ID NO: 17 - DNA, 46 bp, synthetic construct
  source 1..46: mol_type = other DNA
gctcgagaggccttgaattctcagttgtctgtgttggctttagcag

SEQ ID NO: 18 - DNA, 45 bp, synthetic construct
  source 1..45: mol_type = other DNA
caaggaattcaaggcctctcgaatgagcgatcactacgagagagt

SEQ ID NO: 19 - DNA, 40 bp, synthetic construct
  source 1..40: mol_type = other DNA
actcactatagttctagaggctcagttgtctgtgttggct

SEQ ID NO: 20 - DNA, 41 bp, synthetic construct
  source 1..41: mol_type = other DNA
cttgaagcctgtttttattaggagtcaatgccaatcccagc

SEQ ID NO: 21 - DNA, 40 bp, synthetic construct
  source 1..40: mol_type = other DNA
gctgggattggcattgactcctaataaaacaggcttcaag

SEQ ID NO: 22 - DNA, 25 bp, synthetic construct
  source 1..25: mol_type = other DNA
cggctgatttgttatctcatctagg

SEQ ID NO: 23 - DNA, 25 bp, synthetic construct
  source 1..25: mol_type = other DNA
cctcttacctcagttacaatttata